SEQUENCE LISTING (9 sequences)

SEQ ID NO: 1 - DNA, 2059 bp, Artificial Sequence
  Description of Artificial Sequence Synthetic polynucleotide
gggaaataagagagaaaagaagagtaagaagaaatataagagccaccatggccgagaagagaagaggcagcccctgctccatgctgagcctgaaggcccacgccttcagcgtggaagctctgatcggcgctgagaagcagcagcagctgcagaagaagaggcggaagctgggcgccgaagaggccgctagagctgtggatgatggcggctgttctagaggcggcggagctggcgagaagggatcttctgaaggcgacgagggcgctgccctgcctcctcctgctggcgctacatctggccctgctagaagcggcgctgacctggaaagaggcgctgctgggggctgcgaggatggatttcagcagggcgctagcccactggctagccctggcggatctcctaagggcagccctgccagatccctggccagacctggaacacctctgcctagccctcaggcccctagagtggatctgcagggggccgaactgtggaagagattccacgagatcggcaccgaaatgatcatcaccaaggccggcagacggatgttccccgccatgagagtgaagatcagcggcctggacccccaccagcagtactatatcgccatggacatcgtgcccgtggacaacaagagatacagatacgtgtaccacagcagcaagtggatggtggccggcaacgccgactctcccgtgcctcctagagtgtacatccaccctgacagccccgccagcggcgagacatggatgagacaagtgatcagcttcgacaagctgaagctgaccaacaacgagctggacgaccagggccacatcatcctgcacagcatgcacaagtaccagcccagagtgcacgtgatcagaaaggactgcggcgacgacctgagccccatcaagcctgtgcctagcggagagggcgtgaaggctttcagcttccccgagacagtgttcaccaccgtgaccgcctaccagaaccagcagatcaccagactgaagatcgacagaaaccccttcgccaagggcttcagagacagcggcagaaacagaatgggcctggaagccctggtggaaagctacgccttttggaggcccagcctgagaaccctgaccttcgaggacatccccggcatccccaagcagggcaacgccagcagtagcacactgctgcagggaaccggaaacggcgtgccagccacacaccctcatctgctgagcggcagctcttgcagcagcccagcttttcacctgggccccaacaccagccagctgtgttctctggcccctgccgactacagcgcctgtgctagatctggcctgaccctgaacagatacagcaccagcctggccgagacatacaacagactgaccaaccaggccggcgagactttcgcccctcctagaacccctagctacgtgggcgtgtccagcagcacctccgtgaacatgagcatgggcggcaccgacggcgacaccttcagctgtcctcagaccagcctgtccatgcagatctccggcatgagccctcagctgcagtacatcatgcccagccctagcagcaacgccttcgccaccaaccagacacaccagggcagctacaacaccttccggctgcacagcccttgcgccctgtacggctacaacttctccaccagccctaagctggccgccagccccgagaagatcgtgtctagccagggaagctttctgggcagctcccccagcggcaccatgaccgatagacagatgctgccccccgtggaaggcgtgcacctgctgtctagcggaggccagcagagcttcttcgacagcagaaccctgggcagcctgacactgagcagcagccaggtgtccgcccacatggtgcctgccgctgctacagagcagaagctgattagcgaagaggacctggccgctaacgacatcctggactacaaggacgacgacgacaaagtgtgataagctgccttctgcggggcttgccttctggccatgcccttcttctctcccttgcacctgtacctcttggtctttgaataaagcctgagtaggaaggc

SEQ ID NO: 2 - DNA, 1132 bp, Artificial Sequence
  Description of Artificial Sequence Synthetic polynucleotide
gggaaataagagagaaaagaagagtaagaagaaatataagagccaccatggattacggcggagccctgtctgccgtgggcagagaactgctgttcgtgaccaaccccgtggtcgtgaacggcagcgtgctggtgcctgaggaccagtgttactgcgccggctggatcgagagcagaggcacaaacggcgcccagaccgcctctaacgtgctgcagtggctggccgctggcttcagcatcctgctgctgatgttctacgcctaccagacctggaagtctacctgcggctgggaagagatctacgtgtgcgccatcgagatggtcaaagtgatcctggaattcttcttcgaattcaaaaacccctccatgctgtacctggccaccggccacagagtgcagtggctgagatacgccgagtggctgctgacctgccccgtgatctgcatccacctgagcaacctgaccggcctgtccaacgactacagcagacggaccatgggcctgctggtgtccgacatcggcacaatcgtgtggggcgccacaagcgccatggccacaggctacgtgaaagtgatcttcttctgcctgggcctgtgctacggcgccaacacattcttccacgccgccaaggcctacatcgagggctaccacacagtgcccaagggcagatgcagacaggtcgtgacaggcatggcctggctgttcttcgtgtcctggggcatgttccccatcctgttcatcctgggccctgagggcttcggcgtgctgtctgtgtacggctctaccgtgggccacaccatcatcgacctgatgagcaagaactgttggggactgctgggccactacctgagagtgctgatccacgagcacatcctgattcacggcgacatcagaaagaccaccaagctgaacatcggcggcaccgagatcgaggtggaaaccctggtggaagatgaggccgaggctggcgctgtgccagccgctgctacaggtaagcctatccctaaccctctcctcggtctcgattctacgtgataagctgccttctgcggggcttgccttctggccatgcccttcttctctcccttgcacctgtacctcttggtctttgaataaagcctgagtaggaaggc

SEQ ID NO: 3 - DNA, 1777 bp, Artificial Sequence
  Description of Artificial Sequence Synthetic polynucleotide
gggaaataagagagaaaagaagagtaagaagaaatataagagccaccatggaaggcttcgacggcagcgatttttcccctcctgccgatctcgttggagttggcggagccgtgatgagaaacgtggtggacgtgaccatcaacggcgacgtgacagcccctcctaaagccgctcctagaaagtccgagagcgtgaagaaggtgcactggaacgacgtggaccagggacctagcgagaagcccgagacacggcaagaggaaagaatcgacatccccgagatcagcggcctttggtggggagagaatgagcatggtgttggcggcggacggatggaaatccctacaacaggcgtgggcagagtgcagtttagagtgcgggccgtgattgaccacctgggcatgagagcctttggcgtgttcctgatcctgctggacatcatcctgatgatcattgacctgagcctgcctggcaagagcgagagcagccagagcttctatgatggcctggctctggccctgagctgctacttcatgctggatctgggcctgagaatcttcgcctacggacccaagaacttcttcacaaacccctgggaagtcgccgacggcctgatcatcgtggtcacctttgtggtcaccatcttctacaccgtgctggacgagtacttccaagagacaggcgccgatggactgggacagctggttgttctggctcggctgctgagagtcgtcagactggccagaatcttctactcccaccagcagagagtggtgtccgagagaatgtaccccgaggacggcgtgctgaagtctgagatcaagaaaggcctgcggctgaaggacggcggacactatgctgccgtggtcaagaccacctacaaggccaagaaacccgtgcagctgcctggcgcctacatcgtggatatcaagctggacattgtgtcccacaacgaggactacaccatcgtggaacagtgcgagagagccgagggcagacattctacaggcggcatggacgagctgtacaaaggcggaacaggcggctccctggtgtccaaaggcgaggaagtgaacaaggccattatcaaagaattcatgcggttcaaggtccacatggaaggcagcgtgaacggccacgagttcgagattgaaggcgaaggcgagggtagaccctacgaggcctttcagaccgccaagctgaaagtgaccaaaggcggccctctgcctttcgcctgggatatcctgtctcctcagtttatgtacggcagcaaggcctacatcaagcaccccgccgacattcccgactacttcaagctgagcttccccgagggcttcagatgggagagagtgatgaacttcgaggatggcggcatcatccacgtgaaccaggatagctctctgcaggacggggtgttcatctacaaagtgaagctgcggggcaccaactttccacctgatggccctgtgatgcagaaaaagaccatgggctgggaagccaccagacctgccgctgccacatatccctacgacgtgccagactacgcctgatgagctgccttctgcggggcttgccttctggccatgcccttcttctctcccttgcacctgtacctcttggtctttgaataaagcctgagtaggaaggctcgctttcttgctgtccaatttctattaaaggttcctttgttccctaagtccaactactaaactgggggatattatgaagggccttgagcatctggattctgcctaataaaaaacatttattttcattgcgc

SEQ ID NO: 4 - DNA, 1150 bp, Artificial Sequence
  Description of Artificial Sequence Synthetic polynucleotide
gggaaataagagagaaaagaagagtaagaagaaatataagagccaccatgaccgccgtgtctaccacagccacaacagtgctgcaggccacacagagcgacgtgctgcaagagatccagagcaacttcctgctgaacagcagcatctgggtcaacattgccctggccggcgttgtgatcctgctgtttgtggccatgggccgcgatctggaaagccctagagccaagctgatctgggtcgccacaatgctggtgcccctggtgtctatcagcagctatgccggactggcctctggcctgacagtgggctttctgcaaatgcctcctggacacgccctggctggacaagaagttctgtctccctggggcagatacctgacctggaccttcagcacccctatgattctgctggccctgggactgctggccgatacagatatcgccagcctgttcaccgccatcaccatggacatcgggatgtgtgtgacaggcctggccgctgctctgatcacaagcagccatctgctgagatgggtgttctacggaatcagctgcgccttcttcgtggccgtgctgtatgtgctgctggtgcagtggcctgccgatgctgaagctgccggaacctctgagatcttcggcaccctgagaatcctgaccgtggtgctgtggctgggctaccctattctgtttgccctgggctctgaaggcgtggcactgctgtctgtgggagtgacaagctggggctatagcggcctggacatcctggccaaatacgtgttcgcctttctgctcctcagatgggtcgccgccaatgagggaacagtgtctggctctggcatgggcattggatctggcggagctgcccctgctgatgatccagctgctgccacctatccttacgacgtgcccgactacgcctgatgagctgccttctgcggggcttgccttctggccatgcccttcttctctcccttgcacctgtacctcttggtctttgaataaagcctgagtaggaaggctcgctttcttgctgtccaatttctattaaaggttcctttgttccctaagtccaactactaaactgggggatattatgaagggccttgagcatctggattctgcctaataaaaaacatttattttcattgcgc

SEQ ID NO: 5 - DNA, 1176 bp, Artificial Sequence
  Description of Artificial Sequence Synthetic polynucleotide
ttttaagctttaatacgactcactatagggaaataagagagaaaagaagagtaagaagaaatataagagccaccatggattacggcggagccctgtctgccgtgggcagagaactgctgttcgtgaccaaccccgtggtcgtgaacggcagcgtgctggtgcctgaggaccagtgttactgcgccggctggatcgagagcagaggcacaaacggcgcccagaccgcctctaacgtgctgcagtggctggccgctggcttcagcatcctgctgctgatgttctacgcctaccagacctggaagtctacctgcggctgggaagagatctacgtgtgcgccatcgagatggtcaaagtgatcctggaattcttcttcgaattcaaaaacccctccatgctgtacctggccaccggccacagagtgcagtggctgagatacgccgagtggctgctgacctgccccgtgatcctgatccacctgagcaacctgaccggcctgtccaacgactacagcagacggaccatgggcctgctggtgtccgacatcggcacaatcgtgtggggcgccacaagcgccatggccacaggctacgtgaaagtgatcttcttctgcctgggcctgtgctacggcgccaacacattcttccacgccgccaaggcctacatcgagggctaccacacagtgcccaagggcagatgcagacaggtcgtgacaggcatggcctggctgttcttcgtgtcctggggcatgttccccatcctgttcatcctgggccctgagggcttcggcgtgctgtctgtgtacggctctaccgtgggccacaccatcatcgacctgatgagcaagaactgttggggactgctgggccactacctgagagtgctgatccacgagcacatcctgattcacggcgacatcagaaagaccaccaagctgaacatcggcggcaccgagatcgaggtggaaaccctggtggaagatgaggccgaggctggcgctgtgccagccgctgctacaggtaagcctatccctaaccctctcctcggtctcgattctacgtgataagctgccttctgcggggcttgccttctggccatgcccttcttctctcccttgcacctgtacctcttggtctttgaataaagcctgagtaggaaggcggccgcgtcgacaaaaa

SEQ ID NO: 6 - DNA, 1176 bp, Artificial Sequence
  Description of Artificial Sequence Synthetic polynucleotide
ttttaagctttaatacgactcactatagggaaataagagagaaaagaagagtaagaagaaatataagagccaccatggattacggcggagccctgtctgccgtgggcagagaactgctgttcgtgaccaaccccgtggtcgtgaacggcagcgtgctggtgcctgaggaccagtgttactgcgccggctggatcgagagcagaggcacaaacggcgcccagaccgcctctaacgtgctgcagtggctggccgctggcttcagcatcctgctgctgatgttctacgcctaccagacctggaagtctacctgcggctgggaagagatctacgtgtgcgccatcgagatggtcaaagtgatcctggaattcttcttcgaattcaaaaacccctccatgctgtacctggccaccggccacagagtgcagtggctgagatacgccgagtggctgctgacctgccccgtgatctgcatccacctgagcaacctgaccggcctgtccaacgactacagcagacggaccatgggcctgctggtgtccgacatcggcacaatcgtgtggggcgccacaagcgccatggccacaggctacgtgaaagtgatcttcttctgcctgggcctgtgctacggcgccaacacattcttccacgccgccaaggcctacatcgagggctaccacacagtgcccaagggcagatgcagacaggtcgtgacaggcatggcctggctgttcttcgtgtcctggggcatgttccccatcctgttcatcctgggccctgagggcttcggcgtgctgtctgtgtacggctctaccgtgggccacaccatcatcgacctgatgagcaagaactgttggggactgctgggccactacctgagagtgctgatccacgagcacatcctgattcacggcgacatcagaaagaccaccaagctgaacatcggcggcaccgagatcgaggtggaaaccctggtggaagatgaggccgaggctggcgctgtgccagccgctgctacaggtaagcctatccctaaccctctcctcggtctcgattctacgtgataagctgccttctgcggggcttgccttctggccatgcccttcttctctcccttgcacctgtacctcttggtctttgaataaagcctgagtaggaaggcggccgcgtcgacaaaaa

SEQ ID NO: 7 - DNA, 1176 bp, Artificial Sequence; identical to SEQ ID NO: 5
  Description of Artificial Sequence Synthetic polynucleotide

SEQ ID NO: 8 - DNA, 1176 bp, Artificial Sequence; identical to SEQ ID NO: 5
  Description of Artificial Sequence Synthetic polynucleotide

SEQ ID NO: 9 - protein, 8 aa, Artificial Sequence
  Description of Artificial Sequence Synthetic peptide
DYKDDDDK

(19) United States
(12) Patent Application Publication
(10) Pub. No.: US 2021/0000879 A1
(43) Pub. Date: Jan. 7, 2021

(54) METHODS AND COMPOSITIONS FOR MRNA-BASED MODULATION AND DETECTION OF CELL PHENOTYPES

(71) Applicants: Georgia Tech Research Corporation, Atlanta, GA (US); Emory University, Atlanta, GA (US)

(72) Inventors: Philip Santangelo, Atlanta, GA (US); Jonathan Kirschman, Atlanta, GA (US); Hee Cheol Cho, Atlanta, GA (US); David Wolfson, Austin, TX (US); Nam Kyun Kim, Duluth, GA (US); Natasha Fernandez, Atlanta, GA (US)

(21) Appl. No.: 16/976,841

(22) PCT Filed: Mar. 1, 2019
(86) PCT No.: PCT/US19/20219

§ 371 Date: Aug. 31, 2020

Related U.S. Application Data
(60) Provisional application No. 62/637,005, filed on Mar. 1, 2018.

(51) Int. Cl. A61K 35/34; A61K 47/59; A61K 38/18; A61K 38/17; A61K 9/127; A61K 9/51; C12N 5/077

(52) U.S. Cl. CPC A61K 35/34; A61K 47/59; A61K 38/18; A61K 45/06; A61K 9/127; A61K 9/5184; C12N 5/0657; A61K 38/1709

(57) ABSTRACT

Embodiments of the present disclosure relate generally to expression of synthetic messenger RNA (mRNA) in target cells (e.g., a cardiac cell, such as for example and not limitation, a cardiomyocyte, a neuronal cell, a cell located within the eye, a pancreatic cell, a PSC, an IPSC, an ESC, and/or a PSC cardiomyocyte) in order to modulate and/or detect cell phenotype, and more specifically to use of a composition comprising (i) at least one (or a combination of) mRNA(s) encoding a differentiation factor, a transcription factor and/or a phenotype sensor; and (ii) a delivery vehicle, such as for example and not limitation, a cationic lipid, a polyethylenimine (PEI) derivative, a polymer, a polypeptide or peptide, a nanoparticle, or a lipid-based particle, wherein the composition is delivered to the target cell.

### CROSS-REFERENCE TO RELATED APPLICATIONS

This application claims priority to U.S. Provisional Application No. 62/637,005, filed on 1 Mar. 2018, the disclosure of which is herein incorporated by reference in its entirety.

### GOVERNMENT SPONSORSHIP

This invention was made with government support under Grant No. HR0011-16-2-0016 awarded by DARPA and Grant No. 1R01HL111646-01A1 (HL111646) awarded by the National Institutes of Health (National Heart, Lung, and Blood Institute). The government has certain rights in the disclosure.

### BACKGROUND OF THE DISCLOSURE

### 1. Field of the Disclosure

Embodiments of the present disclosure relate generally to expression of messenger RNA (mRNA) in a cell, such as for example and not limitation, cardiac cells, neurons, optical cells, pancreatic cells, and/or pluripotent stem cells (e.g., induced pluripotent stem cells (iPSCs or IPSCs) and embryonic stem cells (ESCs)) in order to modulate and/or detect a cell phenotype, and more specifically to use of a composition comprising (i) at least one (or a combination of) mRNA(s) encoding a differentiation factor, a transcription factor and/or a phenotype sensor; and (ii) a delivery vehicle, such as for example and not limitation, a cationic lipid, a polyethylenimine derivative, a polypeptide or peptide, a nanoparticle, and/or a lipid-based particle, wherein the composition is delivered to the cell, such as for example and not limitation, a model system including cardiac cells and/or PSCs, which can be generated either from a cell line or from ex vivo primary cells.

### 2. Background

Differentiation factors result in cells expressing phenotypes corresponding to ventricular, atrial or pacemaker cardiomyocytes (Kapoor et al, Nature Biotechnology, 2013). Phenotype sensors are used to measure cellular metrics such as muscle contraction force (strain sensitive fluorescent proteins) and membrane potential (voltage-sensitive fluorescent proteins).

The implantation of stem cells for cardiac regeneration, especially after ischemic heart failure, has currently been the subject of many studies and a few human trials. However, to date, studies have focused on implanting different types of stem cells and not on the specific phenotypes of the cells. Also, they have suffered from the inability to assess phenotype, especially after implantation, as well as safety issues in generating the particular phenotype or sourcing the stem cells. The use of mRNA, which has a strong safety profile compared to viral or DNA methods, as well as transient expression, allows both control and monitoring of phenotype. Cells can be sorted based on their expression profile so that cells to be implanted are a homogenous population of the same phenotype. Once damaged heart tissue is “repaired,” myocytes can return to a native state and/or redifferentiate based on the patient-host environment. Furthermore, tissue in vivo can be assessed for phenotype allowing for better understanding of the state of disease tissue both before and after treatment.

The current state-of-the-art model system for in vitro cardiac modeling for simulating diseases and/or testing of therapeutic compounds and formulations involves transfection of cardiac or iPSCs using viral vectors. However, viral vectors limit the amount of testing permutations possible due to (1) slow transfection rates resulting in a 1 to 2 week delay before trials can be performed, (2) the semi-permanent nature of viral transfections, and (3) the inability to express multiple genes in a highly controlled manner. The use of mRNA to express genes allows rapid (1-2 days), transient expression of multiple genes simultaneously in a stoichiometric manner, which can lead to better phenotype control, and the ability to control length of expression through multiple dosing.

Heart rhythm abnormalities lead to over 3 million new cases in the U.S. alone. Existing pharmacotherapies for cardiac arrhythmias often have off-target effects, and attrition rates of new drug development is exceedingly high. Drugs that target other organs need to be proven safe for cardiotoxicity. However, present platforms of cardiac myocytes are largely inadequate for in vitro drug testing. Methods for evaluating cardiomyocytes' function can vary greatly, and thus are difficult to standardize. Human iPSCs can routinely give rise to cardiac myocytes, providing human heart cells at unlimited quantities and bypassing ethical issues of embryonic stem cells (ESCs). Unfortunately, the stem cell-derived cardiomyocytes can be a random mixture of atrial, ventricular and pacemaker myocytes. This inherent heterogeneity is a problem for drug screen and cell therapies for cardiac regeneration.

What is needed, therefore, is an improved method of modifying and/or detecting the phenotypes of cardiac cells, neurons, optical cells, pancreatic cells, and/or PSCs. The method should take advantage of known transfection techniques into these cell types, yet improve transfection efficiency and mRNA expression to provide optimized methods of modulating and detecting cell phenotypes in vivo. Compositions that provide such optimized methods comprise (i) at least one (or a combination of) mRNA(s) encoding a differentiation factor (such as for example and not limitation, TBX18, TBX3, TBX, and SHOX2), a transcription factor, and/or a phenotype sensor (such as for example and not limitation, an opsin such as Quasar, Jaws, Catch-V5, and/or ChR2 or a sensor protein such as Archer1, FlicR1, ArcD95H, GCaMP6f, and/or cTNT-E2Crimson); and (ii) a delivery vehicle, such as for example and not limitation, a cationic lipid (e.g., Lipofectamine), a polyethylenimine (PEI) derivative (e.g., a linear PEI derivative), a polymer (e.g., a virus-like polymer such as ViroRed®) a polypeptide or peptide (e.g., a viral polypeptide or protein), a nanoparticle (e.g., a virus or a virus-like particle), a nanoparticle (e.g., a virus-like particle) and/or a lipid-based particle (e.g., a liposome or nanoliposome), wherein the composition is delivered to a target cell, such as for example and not limitation, a cardiac model system including cardiac cells and/or PSCs, which can be generated either from a cell line or from ex vivo primary cells. The composition can optionally include mRNA encoding proteins which modulate the cellular environment of the target cell or serve as reporters for measuring expression and/or cell phenotype, and/or small molecules tethered to the at least one mRNA. The compositions of the disclosure can also be used to treat cardiac diseases, such as for example and not limitation, atrioventricular block, sick sinus syndrome, bradyarrhythmias and other arrhythmias, and problems initiating a heartbeat from the sinoatrial node which typically require the implantation of a pacemaker device. The compositions of the disclosure can also be used in in vitro cardiac modeling for simulating diseases and/or testing of therapeutic compounds and formulations. It is to such a composition and methods of modulating and/or detecting cell phenotypes that embodiments of the present disclosure are directed.

### BRIEF SUMMARY OF THE DISCLOSURE

As specified in the Background Section, there is a great need in the art to identify technologies for modifying and/or detecting the phenotypes of cardiac cells, neurons, optical cells, pancreatic cells, and/or pluripotent stem cells and use this understanding to develop novel compositions and methods for such modulation and/or detection. The present disclosure satisfies this and other needs. Embodiments of the present disclosure relate generally to optimized methods of modulating and detecting cell phenotypes in vivo, such as for example and not limitation, cell phenotypes in cardiac tissue. Compositions that provide such optimized methods comprise (i) at least one (or a combination of) mRNA(s) encoding a differentiation factor (such as for example and not limitation, TBX18, TBX3, TBX5, and SHOX2), a transcription factor, and/or a phenotype sensor (such as for example and not limitation, an opsin such as for example and not limitation an opsin such as for example and not limitation Quasar, Jaws, Catch-V5, and/or ChR2 or another sensor protein such as for example and not limitation Archer1, FlicR1, ArchD95H, GCaMP6f, and/or cTNT-E2Crimson); and (ii) a delivery vehicle, such as for example and not limitation, a cationic lipid (e.g., a lipofectamine), a polyethylenimine (PEI) derivative (e.g., a linear PEI derivative), a polymer (e.g., a virus-like polymer such as ViroRed®) a polypeptide or peptide (e.g., a viral polypeptide or protein), a nanoparticle (e.g., a virus-like particle) and/or a lipid-based particle (e.g., a liposome or nanoliposome), wherein the composition is delivered to a target cell, such as for example and not limitation, a cardiac model system including cardiac cells and/or PSCs, which can be generated either from a cell line or from ex vivo primary cells. The composition can optionally include mRNA encoding proteins which modulate the cellular environment of the target cell or serve as reporters for measuring expression and/or cell phenotype, and/or small molecules tethered to the at least one mRNA. The compositions of the disclosure can also be used to treat cardiac diseases, such as for example and not limitation, block of electrical flow in the atrial and ventricular conduction system that causes bradyarrhythmias such as atrioventricular block or His bundle block, problems in initiating the heartbeat from the sinoatrial node such as sick sinus syndrome, and other arrhythmias which typically require the implantation of a pacemaker device. The compositions of the disclosure can also be used in in vitro cardiac modeling for simulating diseases and/or testing of therapeutic compounds and formulations.

In one aspect, the disclosure provides a composition comprising: (i) at least one messenger RNA (mRNA) encoding a differentiation factor, a transcription factor, and/or a phenotype sensor; and (ii) a delivery vehicle comprising a cationic lipid, a polyethylenimine (PEI) derivative, a polymer, a polypeptide or peptide, a nanoparticle, or a lipid-based particle.

In another aspect, the disclosure provides a method of modulating cell phenotypes comprising the steps of: (i) formulating a composition comprising: at least one mRNA expression vector encoding a differentiation factor, a transcription factor, and/or a phenotype sensor; and a delivery vehicle comprising a cationic lipid, a polyethylenimine (PEI) derivative, a polymer, a polypeptide or peptide, a nanoparticle, or a lipid-based particle; (ii) administering the composition to a target cell via transfection; (iii) optionally detecting the phenotype of the target cell; and (iv) optionally providing one or more phenotypically appropriate target cells to a patient to treat and/or prevent a disease.

In another aspect, the disclosure provides a method of treating and/or preventing a cardiac disorder in a subject in need thereof comprising the steps of: (i) formulating a composition comprising: at least one messenger RNA (mRNA) encoding a differentiation factor, a transcription factor, and/or a phenotype sensor; and a delivery vehicle comprising a cationic lipid, a polyethylenimine (PEI) derivative, a polymer, a polypeptide or peptide, a nanoparticle, or a lipid-based particle; (ii) administering the composition to a target cell selected from the group consisting of a cardiac cell, a cardiomyocyte, a PSC, an IPSC, an ESC, and a PSC cardiomyocyte via transfection; (iii) optionally detecting the phenotype of the transfected target cell by detecting the activity of the phenotype sensor; and (iv) re-implanting the transfected target cell in the subject, where the subject optionally has an electric pacemaker device.

In another aspect, the disclosure provides a method of determining a cell phenotype comprising: (i) formulating a composition comprising: at least one mRNA expression vector encoding a phenotype sensor; and a delivery vehicle comprising a cationic lipid, a polyethylenimine (PEI) derivative, a polymer, a polypeptide or peptide, a nanoparticle, or a lipid-based particle; (ii) administering the composition to a target cell via transfection; and (iii) detecting the phenotype of the target cell.

These and other objects, features and advantages of the present disclosure will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1A-1B shows specific derivation of AMs, VMs and PMs from hiPSCs. FIG. 1A shows that the retinoic acid-mediated pathway dictated atrial myocytes (A-like hashed area in the RA group) or ventricular myocytes (blank area in the RiRA group). FIG. 1B shows that the Shox2-mediated pathway gave rise to >60% pacemaker myocytes (PMs).

FIG. 2 shows the generation of pacemaker myocyte (PM) spheroids on an Aggrewell plate. Cells were plated to achieve 1000 PMs per spheroid. The spheroids became more compact over time, were viable for >4 weeks, and demonstrated spontaneous and rhythmic pacing.

FIG. 3A-3D shows functional testing of mRNA-expressed opsins in human iPS cell-derived cardiomyocytes on multielectrode array (MEA). (3A) Electrical spike activity from one of three replicate wells transfected with 333 ng of ChR2-mRNA and assayed 24 hours post transfection. (3B) Complete control of beat rate through step-wise increase in the rate of blue excitation light. (3C) Capture success (complete synchronization of beat rate) of ChR2 mRNA-transfected cells out of three replicates. The highest capture success occurs with the highest amount of delivery vehicle and is capable of driving beat rates up to 500 ms. (3D) Functional expression of JAWS-mRNA in cardiomyocytes illustrated by arrest of native spikes upon the illumination of orange light (duplicate wells).

FIG. 4 shows ventricular myocytes expressing CatCh mRNA that were stained for viability, α-SA, and CatCh-V53. Cells were measured using a BD LSRFortessa flow cytometer.

FIG. 5A-5C shows rates and thresholds of NRVM capture over time with increasing amounts of CatCh, 0.1 ng/1000 cells (5A), 1 ng/1000 cells (5B), and 10 ng/1000 cells (5C).

FIG. 6A-6B show sample action potential signals from a single cell transfected with FlicR RNA. (6A) Sample fluorescence signal. (6B) Average action potential.

FIG. 7 shows excitation intensity comparison between ChR2 and CatCH mRNA-transfected NRVMs. NRVMs were transfected one day after plating on MEA plates with either ChR2 or CatCH mRNA. The sensitivity of the opsin to excitation light is inversely proportional to the percent LED power required to evoke a response. CatCH shows the highest sensitivity at 24 hours post transfection, where it responds to 5% intensity LED light. This is 15 times the LED power required for ChR2.

FIG. 8 shows maximum beat rate comparison between ChR2 and CatCH mRNA-transfected NRVMs. NRVMs were transfected one day after plating on MEA plates with either ChR2 or CatCH mRNA. They were driven at 1 hz, 2 hz, and 3 hz via LED pulsing. Responses were recorded here if all 3 replicate wells were able to beat at the indicated rates using maximum intensity excitation light. CatCH was able to sustain higher frequencies throughout the time course.

FIG. 9 shows iPS derived cardiomyocytes transfected with mRNA encoding ChR2 and JAWS; here both excitation (arrows) and inhibition (bars) are demonstrated.

FIG. 10 shows transgene expression using the mRNA expression systems described herein was transient evidenced by the weaker GFP signal over time.

FIG. 11 shows that TBX18 IVT mRNA successfully and efficiently enters cardiac myocytes in a transient manner.

FIG. 12 shows that TBX18 IVT mRNA gene delivery leads to efficient and transient expression of TBX18 protein in cardiac myocytes.

FIG. 13A-13C shows that TBX18 IVT mRNA gene delivery leads to correct localization of TBX18 proteins to cardiac myocytes' nuclei. 13A. 1 μg of IVT mRNA+0.2 μl of ViroMer Red. 13B. 0.5 μg of IVT mRNA+0.2 μl of ViroMer Red. 13C. 1 μg of IVT mRNA+0.1 μl of ViroMer Red. Cardiomyocytes positive for nuclear TBX18 (FLAG positive) were 21%, 4% and 2% for 13A, 13B and 13C, respectively. (scale bar: 50 μm)

FIG. 14A-14B shows that TBX18 IVT mRNA gene transfer converts ordinary heart muscle cells to de novo pacemaker cells (14B) whereas a control does not (14A).

FIG. 15A-15C shows that direct myocardial injection of IVT mRNA achieves focal cardiac gene delivery in vivo. (15A) Myocardium before injection. (15B) Myocardium during injection. (15C) Myocardium after injection.

FIG. 16 shows that TBX18 mRNA creates ventricular pacing in a rat model of complete heart block.

FIG. 17A-17B shows that TBX18 mRNA gene transfer creates ventricular pacing in a rat model of complete heart block via continuous recording of the animals' heart rate (17A) and heart rate histograms (17B).

FIG. 18 shows that TBX18 mRNA creates ventricular pacing in a porcine model (days 7-10 after biologic delivery).

FIG. 19 shows that TBX18 mRNA with A83-01 creates ventricular pacing in a porcine model (day 10 after biologic delivery).

### DETAILED DESCRIPTION OF THE DISCLOSURE

As specified in the Background Section, there is a great need in the art to identify technologies for modifying and/or detecting the phenotypes of cardiac cells, neurons, optical cells, pancreatic cells, and/or PSCs (including IPSCs and ESCs) and use this understanding to develop novel compositions and methods for such modulation and/or detection. The present disclosure satisfies this and other needs. Embodiments of the present disclosure relate generally to optimized methods of modulating and detecting cell phenotypes in vivo, such as for example and not limitation, in cardia tissue. Compositions that provide such optimized methods comprise (i) at least one (or a combination of) mRNA(s) encoding a differentiation factor (such as for example and not limitation, TBX18, TBX3, TBX5, and SHOX2), a transcription factor, and/or a phenotype sensor (such as for example and not limitation, Quasar, Jaws, Catch-V5, ChR2, FlicR1, Archer1, ArchD95H, GCaMP6f, and/or cTNT-E2Crimson); and (ii) a delivery vehicle, such as for example and not limitation, a cationic lipid (e.g., Lipofectamine), a polyethylenimine (PEI) derivative (e.g., a linear PEI derivative), a polymer (e.g., a virus-like polymer such as ViroRed®) a polypeptide or peptide (e.g., a viral polypeptide or protein), a nanoparticle (e.g., a virus-like particle) and/or a lipid-based particle (e.g., a liposome or nanoliposome), wherein the composition is delivered to a target cell, such as for example and not limitation, a cardiac model system including cardiac cells and/or PSCs, which can be generated either from a cell line or from ex vivo primary cells. The composition can optionally include mRNA encoding proteins which modulate the cellular environment of the target cell or serve as reporters for measuring expression and/or cell phenotype, and/or small molecules tethered to the at least one mRNA. The compositions of the disclosure can also be used to treat cardiac diseases, such as for example and not limitation, a block of electrical flow in the atrial and ventricular conduction system that causes bradyarrhythmias such as atrioventricular block or His bundle block, problems in initiating the heartbeat from the sinoatrial node such as sick sinus syndrome, and other arrhythmias which typically require the implantation of a pacemaker device. The compositions of the disclosure can also be used in in vitro cardiac modeling for simulating diseases and/or testing of therapeutic compounds and formulations.

The present invention provides an approach for the novel use of synthetic mRNA to modulate and detect cell phenotype in in vitro systems, such as cardiac model systems. The approach comprises the use of the compositions as discussed herein, along with transfecting such mRNAs into target cells such as cardiac cells, neurons, optical cells, pancreatic cells, and/or PSCs. The use of mRNA to express genes allows rapid (1-2 days), transient expression of multiple genes simultaneously in a stoichiometric manner, which can lead to better phenotype control, and the ability to control length of expression through multiple dosing.

The inventors have optimized mRNA expression in cardiac cells with very high transfection efficiency (>98%). The inventors have also demonstrated expression of TBX18, a differentiating factor, using mRNA to induce neonatal rat ventricular myocytes to express pacemaker cell phenotypes in vitro, as well as other differentiation factors. This has led to the observation and quantification of contraction behavior using imaging. The inventors have also expressed mRNA successfully in PSC-derived cardiomyocytes and observed electrical activity using multi electrode arrays. Combining the electrical readout with imaging modalities to assess phenotype creates a highly robust system to assess and optimize pharmaceutical compounds in the R&D phase. Expression of phenotype sensors and differentiating genes in vivo opens up new methods of diagnosis and treatment for heart disorders as well as provides a tool to better understand stem cell function post-implantation.

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present disclosure are intended to be illustrative and not restrictive. Many suitable materials that would perform the same or a similar function as the materials described herein are intended to be embraced within the scope of the disclosure. Such other materials not described herein can include, but are not limited to, materials that are developed after the time of the development of the disclosure, for example. Any dimensions listed in the various drawings are for illustrative purposes only and are not intended to be limiting. Other dimensions and proportions are contemplated and intended to be included within the scope of the disclosure.

As used herein, the term “subject” or “patient” refers to mammals and includes, without limitation, human and veterinary animals. In a preferred embodiment, the subject is human.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or analogues administered as well as the disease and its severity and the age, weight, physical condition and responsiveness of the mammal to be treated.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

As used herein, the term “combination” of a composition according to the present disclosure and at least a second pharmaceutically active ingredient means at least two, but any desired combination of compounds can be delivered simultaneously or sequentially (e.g., within a 24 hour period). Within the meaning of the present disclosure, the term “conjoint administration” is used to refer to administration of a composition according to the disclosure and another therapeutic agent simultaneously in one composition, or simultaneously in different compositions, or sequentially (preferably, within a 24 hour period).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Compositions of the Disclosure

Compositions according to the present disclosure include nucleic acids, expression vectors comprising such nucleic acids, and cells comprising either the nucleic acid or expression vector.

In one aspect is provided a nucleic acid which is a messenger RNA (mRNA) molecule. In some embodiments, the mRNA molecule comprises a differentiation factor, such as for example and not limitation, TBX18, TBX3, TBX5, and SHOX2. In some embodiments, the mRNA comprises a transcription factor. In some embodiments, the mRNA comprises a phenotype sensor, such as for example and not limitation, an opsin or a protein that can detect phenotypic changes in a cell, such as for example and not limitation, voltage changes, calcium changes, and contractility or motility. Non-limiting exemplary opsins include Quasar, Jaws, Catch-V5, and/or ChR2. Non-limiting exemplary sensor proteins include FlicR1, Archer1, ArchD95H (red-shifted voltage-sensor protein), GCaMP6f (green-shifted Ca2+ sensor protein), and/or cTNT-E2Crimson (far red-shifted cardiac contractility protein). In some embodiments, a combination of mRNAs comprising different differentiation factors and/or transcription factors and/or phenotype sensors are transfected into the same cell(s).

In one aspect is provided an mRNA expression vector for the expression of a differentiation factor, a transcription factor, and/or a phenotype sensor wherein the mRNA expression vector comprises a nucleic acid sequence encoding a differentiation factor, a transcription factor, and/or a phenotype sensor as described herein. The mRNA expression vector may comprise a heterologous promoter, such as for example and not limitation a T7 promoter. The mRNA expression vector may comprise a Kozak sequence. Those of ordinary skill appreciate that any promoter or regulatory sequence may be chosen to provide suitable expression of the differentiation factor, transcription factor, and/or phenotype sensor. Without wishing to be bound by theory, the inventors have found that mRNA provides a superior way to express such proteins in a cell for further testing (such as on the ability to induce differentiation of a PSC into a desired cell type and to specifically identify that cell type, or on the electrical activity of a cell in response to light stimulation). The protein can be expressed by the mRNA expression vector in as little as three hours, with expression able to persist for seven days. The various modes of mRNA expression described have minimal impact on the normal functioning of a cell, for example by not introducing a virus into the cell. In some embodiments, mRNA expression vectors encoding different differentiation factors and/or transcription factors and/or phenotype sensors are transfected into the same cell(s).

In some embodiments, the differentiation factor is a T-box transcription factor encoding gene (Tbx gene). Non-limiting examples of Tbx genes include Tbx18, Tbx3, and Tbx5. In some embodiments, the differentiation factor is a special homeobox protein. Non-limiting examples of special homeobox proteins include SHOX2.

In some embodiments, the phenotype sensor is an opsin. In some embodiments, the opsin is an excitatory opsin. In some embodiments, the opsin is an inhibitory opsin. Non-limiting examples of excitatory opsins include Quasar, Channel-rhodopsin II (ChR2) and CatCh, which is activated with pulsed blue light. Non-limiting examples of inhibitory opsins include Jaws, which is activated by constant orange or red light. Other examples of opsins include, but are not limited to, Type I opsins (e.g., bacteriorhodopsin, xanthorhodopsin, halorhodopsin, rhodopsin I, rhodopsin B, channelrhodopsin (ChR), an archaerhodopsin (Arch), Type II opsins (e.g., ciliary opsins, pinopsin, rhodopsin (Rh1), long-wavelength sensitive (OPN1LW) opsin, middle-wavelength sensitive (OPN1MW) opsin, short-wavelength sensitive (OPN1SW) opsin, parapinospin, parietopsin, panopsin (OPN3), teleost multiple tissue (TMT) opsin, r-opsin, melanopsin, Go-opsin, RGR opsin, peropsin, and neuropsin.)

In some embodiments, the phenotype sensor is a protein that can detect changes in cell electrophysiology, including but not limited to membrane potential, intracellular calcium handling or physiology of organelles such as mitochondria. Non-limiting examples of such proteins include FlicR1, Archer1, ArchD95H (red-shifted voltage-sensor protein), GCaMP6f (green-shifted Ca2+ sensor protein), and/or cTNT-E2Crimson (far red-shifted cardiac contractility protein). Other voltage indicator proteins that may be used include, but are not limited to, FlaSH, VSFP1, SPARC, VSFP2, Flare, VSFP3.1, Mermaid, hVOS, PROPS, ArcLight, Arch, ElectricPk, VSFP-Butterfly, VSFP-CR, Mermaid2, Mac GEVI, QuasAr1, QuasAr2, Archer, ASAP1, Ace GEVI, Pado, and ASAP2f.

In some embodiments, the mRNA expression vector is capped. The cap can comprise any cap architecture. For example, the mRNA can be capped by enzymatic N7-methyl guanosine (m7G) capping of the 5′ triphosphate end, creating cap 0. Cap 0 can undergo further methylation on the ribose 2′-hydroxyl (2′-O) of the first nucleoside in a reaction catalyzed by RNA 2′-O-ribose methyltransferase to produce the cap 1 structure. Cap1 structures that contain a 5′ terminal 2′-O methylated adenosine can undergo further methylation at the N6 position. Further methylation of cap 1 at the ribose 2′-O position of the second nucleoside by similar enzymes results in formation of the cap 2 structure. To form the cap 4 architecture, the first nucleoside (adenine) of the transcript undergoes further methylation at the N6 and ribose 2′-O positions in reactions catalyzed by an as yet uncharacterized enzyme and a ribose 2′-O specific methyltransferase (TbMTr1), respectively, to generate 6,6,2 trimethyl adenine. The following three nucleosides (uracils) are subsequently methylated by two additional ribose 2′-O specific methyltransferases (TbMTr2 and TbMtr3) at ribose 2′-O positions to complete cap 4 synthesis. Such capping may reduce the ability of retinoic acid-inducible gene I (RIG-D, or other members of the RIG-I-like receptor (RLR) protein family, to sense the mRNA and provoke an undesirable innate immune response. For example, without capping at the 5′ triphosphate end, RIG-I could rapidly sense and degrade the mRNA. With such capping, the mRNA expression vector can evade detection by RIG-I and other members of the RLR protein family. In various embodiments, capping of the 5′ triphosphate end of the mRNA expression vector is effective for the mRNA expression vector to express the desired protein for at least 3 days, at least 4 days, at least 5 days, and at least 6 days after transfection into a cell comprising RIG-I or a member of the RLR protein family.

In some embodiments, modified nucleosides are incorporated into the mRNA, wherein incorporation of the modified nucleosides is effective to reduce RNA-dependent protein kinase (PKR) and 2′-5′-oligoadenylate synthetase (OAS) activation. The modified nucleoside can include one or more of 2-thiouridine, 5-methyl cytidine (5meC), and pseudouridine (e.g., N1-methyl pseudouridine). In some embodiments, the mRNA comprises both 5meC and pseudouridine. In some embodiments, the modified nucleoside is diaminopurine (DAP), N6-methyl-2-aminoadenosine (me6DAP), N6-methyladenosine (me6A), 5-carboxycytidine (5caC), 5-formylcytidine (5fC), 5-hydroxycytidine (5haC), 5-hydroxymethylcytidine (5hmC), 5-methoxycytidine (5maC), 5-methylcytidine (5meC), N4-methylcytidine (me4C), thienoguanosine (tyG), 5-carboxymethylesteruridine (5camU), 5-formyluridine (5fU), 5-hydroxymethyluridine (5hmU), 5-methoxyuridine (5moU), or 5-methyluridine (5meU).

In some embodiments, the mRNA molecule comprises a polyA tail. A polyA tail may be added to the mRNA molecule. A polyA tail already present on in the mRNA molecule may be increased in length. As a non-limiting example, the polyA tail may be enzymatically added. The polyA tail may range from 1 to 1200 bases in length. The polyA tail may be from 1-75 bases in length, 75-100 bases in length, 85-110 bases in length, 100-125 bases in length, 110-135 bases in length, 125-150 bases in length, 135-160 bases in length, 150-175 bases in length, 170-220 bases in length, 175-225 bases in length, 200-250 bases in length, 225-275 bases in length, 250-300 bases in length, 275-325 bases in length, 300-350 bases in length, 325-375 bases in length, 350-400 bases in length, 375-425 bases in length, 400-450 bases in length, 425-475 bases in length, 450-500 bases in length, 475-525 bases in length, 500-550 bases in length, 550-600 bases in length, 600-650 bases in length, 650-700 bases in length, 700-750 bases in length, 750-800 bases in length, 800-850 bases in length, 850-900 bases in length, 900-950 bases in length, 950-1000 bases in length, 1000-1050 bases in length, 1050-1100 bases in length, 1100-1150 bases in length, or 1150-1200 bases in length.

In some embodiments, the mRNA molecule comprises at least one untranslated region (UTR) at either or both of the 5′ or 3′ end of the molecule. In some embodiments, the UTR comprises a binding site, e.g., for an RNA-binding protein, and/or a microRNA site.

In some embodiments, the mRNA molecule comprises at least one nucleotide mutation that results in codon optimization of the encoded polypeptide.

In some embodiments, the mRNA molecule is combined with a small molecule. In some embodiments, the small molecule is an inhibitor of TGF-beta signaling, e.g., A83-01. In some embodiments, the small molecule is an inhibitor of an innate immune sensor.

In some embodiments, the mRNA molecule comprises a nucleotide sequence comprising any of SEQ ID NOs: 1-8.

In some embodiments, the mRNA expression vector is packaged in a delivery vehicle such as for example and limitation, a delivery vehicle comprising a cationic lipid, a polyethylenimine derivative, a polypeptide or peptide, a polymer, a nanoparticle, and/or a lipid-based particle. In some embodiments, the cationic lipid is Lipofectamine. In some embodiments, the polyethylenimine (PEI) derivative is a linear PEI derivative. In some embodiments, the polymer is a virus-like polymer such as ViroRed®. In some embodiments, the polypeptide or peptide is a viral polypeptide or protein. In some embodiments, the nanoparticle is a virus-like particle. In some embodiments, the mRNA expression vector is packaged in a lipid-based particle such as a liposome. The liposome may be a nanoliposome. In some embodiments, the mRNA expression vector is packaged in a nanoparticle. The nanoparticle may be a lipid nanoparticle.

In some embodiments, the mRNA expression vector is purified before transient transfection. Purification may be performed by high pressure liquid chromatography (HPLC). Purification, such as by HPLC, may allow for one or both of a reduction in immune activation, an increase in translational potential, and a reduction in TLR signaling in cell culture.

In another aspect is provided a cell that comprises an mRNA expression vector wherein the mRNA expression vector comprises a nucleic acid sequence encoding a differentiation factor, a transcription factor, and/or a phenotype sensor. In some embodiments, the cell is a cardiac cell, such as for example and not limitation, a cardio myocyte. In some embodiments, the cell is a neuronal cell. In some embodiments, the cell is found within the eye. In some embodiments, the cell is a pancreatic cell. In some embodiments, the cell is an PSC, e.g., an IPSC or an ESC. In some embodiments, the cell is a PSC cardiomyocyte. In some embodiments, the cell comprises a second mRNA expression vector comprising a nucleic acid sequence encoding a voltage indicator protein, such as FlicR.

In any of the foregoing, the compositions can be administered in a therapeutically effective amount, alone or in combination with a pharmaceutically acceptable carrier and/or a second therapeutic drug, to treat and/or prevent an arrythmia or improper cardiac pacing or a disorder associated with an arrythmia or improper cardiac pacing, such as for example and not limitation, block of electrical flow in the atrial and ventricular conduction system that causes bradyarrhythmias such as atrioventricular block or His bundle block, and/or problems in initiating the heartbeat from the sinoatrial node such as sick sinus syndrome. In some embodiments, compositions of the disclosure may be provided to a subject who has an electric pacemaker device.

### Methods of the Disclosure

In another aspect is provided a method for transiently expressing a differentiation factor, a transcription factor, and/or a phenotype sensor in a cell comprising introducing an mRNA expression vector for the expression of a differentiation factor, a transcription factor, and/or a phenotype sensor into the cell. In some embodiments, the introducing step comprises transiently transfecting the cell with the mRNA expression vector.

In one aspect is provided a method for modulating cell phenotypes in order to produce a desired cell type that can be used in therapeutic applications. For example and not limitation, a cardiomyocyte or PSC can be differentiated into a cardiac pacemaker cell, which can be used as a biologic pacemaker and provided to a subject in need thereof. These biologic pacemakers can be used to treat various cardiac conditions, such as for example but not limitation arrythmias or improper cardiac pacing, block of electrical flow in the atrial and ventricular conduction system that causes bradyarrhythmias such as atrioventricular block or His bundle block, and/or problems in initiating the heartbeat from the sinoatrial node such as sick sinus syndrome. In some embodiments, the biologic pacemaker can be provided to a subject with an existing electric pacemaker. In some embodiments, the biologic pacemaker can be provided to a subject without an existing electric pacemaker. In some embodiments, the biologic pacemaker can be provided to a subject conjointly with a second therapeutic that also treats similar cardiac conditions. In some embodiments, the phenotype of the resulting differentiated cell can be determined using the methods for detecting cell phenotype as described herein.

In some embodiments, the method comprises transiently expressing a mRNA comprising a differentiation factor or a transcription factor in order to modulate the phenotype of the target cell. Non-limiting exemplary mRNAs comprising a differentiation factor can comprise the nucleic acid sequence comprising SEQ ID NO: 1. In some embodiments, the target cell is a cardiomyocyte and the method comprises modulating the phenotype of the cardiomyocyte such that the cardiomyocyte becomes a cardiac pacemaker cell and is capable of performing all the functions of a cardiac pacemaker cell, including initiating and maintaining heart rhythms. In some embodiments, the target is an PSC and the method comprises modulating the phenotype of the PSC such that it differentiates into a desired cell type. In some embodiments, the cells derived from the PSC are homogenous, meaning that they are of a single subtype. For example and not limitation, an mRNA expression vector as described herein can be transfected into a cardiomyocyte or an IPSC in order to cause the cardiomyocyte to differentiate into an atrial myocyte or a ventricular myocyte or a cardiac pacemaker cell. In some embodiments, the resulting cells can be homogenous, meaning that the cells are either atrial myocytes or ventricular myocytes. In some embodiments, the cells can be phenotyped and classified using the methods for detecting cell phenotypes as described herein.

In some embodiments, the mRNA expression vector comprises a nucleic acid sequence encoding a differentiation factor or a transcription factor. In some embodiments, the differentiation factor is a T-box transcription factor encoding gene (Tbx gene). Non-limiting examples of Tbx genes include Tbx18, Tbx3, and Tbx5. In some embodiments, the differentiation factor is a special homeobox protein. Non-limiting examples of special homeobox proteins include SHOX2 (Sbx2). In some embodiments, different mRNA expression vectors comprising nucleic acid sequences encoding different differentiation and/or transcription factors are transiently transfected into the same cell. In some embodiments, a second mRNA expression vector comprising a nucleic acid sequence encoding a phenotype sensor, such as an opsin (e.g., Quasar, Jaws, Catch-V5, and/or ChR2) or a sensor protein (e.g., FlicR1, Archer1, ArchD95H, GCaMP6f, and/or cTNT-E2Crimson), is transiently transfected into the cell.

In one aspect is provided a method of detecting cell phenotypes, such as for example and not limitation, cell electrophysiology of membrane potential, intracellular calcium handling or physiology of organelles such as mitochondria. In one embodiment, a cardiac pacemaker cell resulting from differentiation from a cardiomyocyte or PSC, as performed by methods described herein, can be classified into an atrial myocyte or a ventricular myocyte or a cardiac pacemaker cell. In another embodiment, the cardiac toxicity of a potential drug is screened in a cell expressing one or more opsins or other sensor proteins as described herein.

In some embodiments, the mRNA expression vector comprises a nucleic acid sequence encoding sensor protein which is an opsin. Non-limiting exemplary mRNAs comprising an opsin can comprise the nucleic acid sequences comprising SEQ ID NOs: 2, 4, 6, 7, and/or 8. In some embodiments, the opsin is an excitatory opsin. In some embodiments, the opsin is an inhibitory opsin. In some embodiments, the opsin is Quasar, Jaws, Catch-V5, and/or ChR2. Non-In some embodiments, the mRNA expression vector encodes a nucleic acid sequence encoding a sensor protein that enables detection of a physiological state of a cell, such as for example and not limitation, the cell's electrophysiology. Non-limiting exemplary mRNAs comprising such sensor proteins can comprise the nucleic acid sequences comprising SEQ ID NOs: 3 and 5. In some embodiments, the sensor protein enables detection of changes in cell voltage, membrane potential, and ion concentration (e.g., Ca2+, Na+, K+). In some embodiments, the sensor protein is FlicR1, Archer1, ArchD95H, GCaMP6f, and/or cTNT-E2Crimson.

In some embodiments, a second mRNA expression vector comprising a nucleic acid sequence encoding a second sensor protein is transiently transfected into the cell.

In some embodiments, transient transfection comprises electroporation. In some embodiments, transient transfection comprises lipofection. In some embodiments, transient transfection comprises modified PEI-mediated delivery, such as for example and not limitation, a linear PEI derivative like JET-PEI. In some embodiments, transient transfection comprises complexing the mRNA with a virus-like polymer (e.g., Viromer® Red). In some embodiments, lipofection is undertaken with modified mRNA (e.g., mRNA modified with 5meC and pseudouridine), which is effective to improve expression of transcripts from the mRNA in the cell.

In some embodiments, other delivery methods and vehicles are used, including a delivery vehicle comprising a cationic lipid, a polyethylenimine derivative, a polypeptide or peptide, a polymer, a nanoparticle, and/or a lipid-based particle. In some embodiments, the cationic lipid is a lipofectamine. In some embodiments, the polyethylenimine (PEI) derivative is a linear PEI derivative. In some embodiments, the polymer is a virus-like polymer such as ViroRed®. In some embodiments, the polypeptide or peptide is a viral polypeptide or protein. In some embodiments, the nanoparticle is a virus-like particle. In some embodiments, the mRNA expression vector is packaged in a lipid-based particle such as a liposome. The liposome may be a nanoliposome. In some embodiments, the mRNA expression vector is packaged in a nanoparticle. The nanoparticle may be a lipid nanoparticle.

In some embodiments, modified nucleosides are incorporated into the mRNA expression vector, wherein incorporation of the modified nucleosides is effective to reduce RNA-dependent protein kinase (PKR) and 2′-5′-oligoadenylate synthetase (OAS) activation. The modified nucleoside can include one or more of 2-thiouridine, 5-methyl cytidine (5meC), and pseudouridine (e.g., N1-methyl pseudouridine). In some embodiments, the mRNA comprises both 5meC and pseudouridine. In some embodiments, the modified nucleoside is diaminopurine (DAP), N6-methyl-2-aminoadenosine (me6DAP), N6-methyladenosine (me6A), 5-carboxycytidine (5caC), 5-formylcytidine (5fC), 5-hydroxycytidine (5haC), 5-hydroxymethylcytidine (5hmC), 5-methoxycytidine (5maC), 5-methylcytidine (5meC), N4-methylcytidine (me4C), thienoguanosine (tyG), 5-carboxymethylesteruridine (5camU), 5-formyluridine (5fU), 5-hydroxymethyluridine (5hmU), 5-methoxyuridine (5moU), or 5-methyluridine (5meU). In various embodiments, the mRNA expression vector localizes to the cellular membrane of the cell in which the mRNA expression vector is transiently expressed.

In another aspect is provided a method for functionally characterizing a cell expressing any of the mRNA expression vectors for the expression of opsins or other sensor proteins described herein. The method may comprise performing multi-electrode array (MEA) analysis on the cell expressing the mRNA expression vector. MEA analysis can also be performed on a similar cell not expressing the mRNA expression vector as a control. MEA analysis may comprise detection of field potential, for example spiking activity in a cell as described herein. MEA analysis may be performed on a device that can simultaneously measure and provide light excitation to samples in multiple compartments, such as a Maestro® device from Axion Biosystems that allows for simultaneous measurement and light excitation of 16 electrodes per well in a 48 well plate. The method may comprise measuring action potential profiles and/or Ca2+ transient dynamics by introducing an intracellular Ca2+ dye and taking time-lapsed photographs of the transfected cells with a suitable camera and imaging suite. The method may further comprise measuring maximum velocities of contraction and relaxation in myocytes by fluorescent cTNT.

In another aspect is provided a method for screening drugs and/or testing one or more drugs for their effect on functional characterization of a cell expressing a phenotype sensor. The drug may be applied to a cardiac cell (such as for example and not limitation, a cardiomyocyte), a neuronal cell, a cell is found within the eye, a pancreatic cell, a PSC (e.g., an IPSC or an ESC), and/or a PSC cardiomyocyte. The drug screening may be used to find drugs that are candidates for treating a cardiac disorder, a neural disorder, an eye disorder, and/or a pancreatic disorder. The method may comprise performing multi-electrode array (MEA) analysis on a cell expressing the mRNA expression vector in which the drug for screening or testing is applied. MEA analysis can also be performed on a similar cell expressing the mRNA expression vector but without the drug applied. MEA analysis may comprise detection of field potential, for example spiking activity in a cell. MEA analysis may be performed on a device that can simultaneously measure and provide light excitation to samples in multiple compartments, such as a Maestro® device from Axion Biosystems that allows for simultaneous measurement and light excitation of 16 electrodes per well in a 48 well plate. The functional expression of a sensor protein such as an opsin (e.g., ChR2) can be measured as a synchronization event between electrical activity and pulsed light excitation from LEDs embedded in the MEA device. If the cell is a cardiac cell, MEA analysis can comprise testing the beat rate or the electrical response in a quantitative manner. For example, the cardiac cells expressing the mRNA expression vector can be paced at various beat rates such as 1 Hz, 2 Hz or 3 Hz. Also, different light patterns from the MEA device can be used to simulate an arrhythmia or another anomaly. The method may comprise measuring action potential profiles and/or Ca2+ transient dynamics by introducing an intracellular Ca2+ dye and taking time-lapsed photographs of the transfected cells with a suitable camera and imaging suite. The method may further comprise measuring maximum velocities of contraction and relaxation in myocytes by fluorescent cTNT.

In some embodiments, the method is for screening drugs and/or testing one or more drugs for their effect on arrhythmia in a cardiac cell expressing a sensor protein, such as for example and not limitation, an opsin. Without wishing to be bound by theory, numerous pharmacotherapies suffer from arrhythmogenic side effects. Assessment of whether a drug may increase or exacerbate arrhythmia is needed for at least the reason that over 1% of patients prescribed class III antiarrhythmic agents suffer from Torsades de Pointes, a highly fatal condition. A better understanding of which drugs are risky for such patients can reduce fatalities.

The method can provide for current methods of studying the properties of target cells, such as for example and not limitation, primary or stem cell-derived cardiomyocytes, in a real-time longitudinal study. The method may comprise transfecting at least one sensor protein into a cardiac cell, e.g., an opsin, and at least one second sensor protein that can measure or indicate the cell electrophysiology (e.g., voltage). The opsins can convert visible light into an electrical response and the voltage indicator protein can exhibit a detectable change in fluorescent intensity in response to a voltage change. Transfection of both the voltage indicator protein and the opsin can be undertaken with a delivery vehicle as described herein, such as for example and not limitation, a modified PEI, such as a linear PEI, or a polymer such as a virus-like polymer (e.g., Viromer Red®). The localization of the expressed proteins may be assayed by microscopy and/or flow cytometry.

In various embodiments, the function of transfected cells is evaluated. The transfected cell may be exposed to a range of optical intensities of light that is capable of stimulating the opsin and/or to a range of conditions that stimulate the sensor protein. For example, CatCH may be stimulated using 475 nm blue light. Various stimulation durations may be used, from 0.5 to 30 milliseconds, from 1 to 15 milliseconds, from 1 to 5 milliseconds, from 4 to 10 milliseconds, from 10 to 15 milliseconds, or from 15 to 30 milliseconds. Monophasic square waves can be used in a train at various frequencies.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1. In Vivo Use of TBX18 Encoding mRNA to Produce New Pacemaker (Sinoatrial Node) Cells in a Rat Heart Block Model

In this experiment, the inventors created a complete atrioventricular block (CAVB) model in rats. The rats were monitored for one week to confirm stable and persistent CAVB.

Materials and Methods

On Day 0, an osmotic pump releasing A83-01 (a TGF-beta inhibitor, Sigma Aldrich) was implanted into the rats. On Day 1, TBX18 mRNA was injected into the rats and a telemetry device was also implanted. Two doses of TBX18 mRNA were given to the rats: (i) a low dose of 100 μg TBX18 mRNA with 68.4 μl RNA-free phosphate-buffered saline (PBS), or (ii) a high dose of 300 μg TBX18 mRNA with 205 μl RNA-free PBS. A continuous telemetry recording of each rat's electrocardiogram (ECG) was made for 21 days after implantation of the telemetry device. In addition, 3-lead surface ECGs were performed on anesthetized animals on Days 7, 14, and 21 along with an isoproterenol challenge. The heart was harvested from each animal on Day 21.

### Example 2. Development of a mRNA Encoding a Sensor Protein

Human induced pluripotent stem cells (hiPSCs) can give rise to de novo cardiac myocytes, but the resultant myocytes are often a random mixture of atrial, ventricular and nodal pacemaker myocytes. This is a problem that impedes the progress of drug screen assays and cell therapies for cardiac regeneration. The absence of multi-modal, real-time, functional readouts for the nascent cardiomyocytes can further compound the problem. This Example is intended to address both problems by i) specific derivation of cardiac myocytes of ventricular, atrial or pacemaker lineages, and ii) all-optical measurements of the myocytes' function powered by in vitro transcribed (IVT), modified mRNA technology.

Abnormally fast or slow heart rhythm affects millions of people worldwide and leads to >3 million new cases in the U.S. alone. Existing pharmacotherapies for cardiac arrhythmias are fraught with off-target effects, and attrition rates of new drug development is exceedingly high. Drugs designed to target other organs need to be proven safe for cardiac toxicity. However, present platforms of cardiomyocytes are largely inadequate. Primary cardiomyocytes from rodents are often difficult to culture, and often suffer from species-specific differences in their electrophysiology. Human iPSCs can provide an unlimited supply of de novo cardiac myocytes, but the derived myocytes consist of random mixtures of atrial, ventricular and nodal myocytes. This heterogeneity is a major cause for false positive/negative drug screen results. In some cases, false negative cardiac safety/toxicity data could mask unwanted ventricular tachycardia, which often precipitates to life-threatening events. Compounding the problem is the lack of a reliable, multimodal, real-time readouts for the cardiomyocytes' function, which hinges on two essential properties of the myocytes; electrical excitation and the subsequent contraction. Present modalities of cardiac myocyte function measurement are good, but can be either single-modality or invasive, and thus incapable of measuring the myocytes' properties at multiple time points in long-term.

This Example describes how the inventors have harnessed two powerful technologies to develop and standardize stem cell-derived cardiomyocyte manufacturing. A small molecule-based technology was exploited to derive subtype-specific cardiac myocytes from hiPSC lines. The de novo human cardiomyocytes displayed electrical and contractile functions tailored to their specific subtype, and their genotype/phenotype were stable for >6 weeks in 2D cultures. An IVT RNA technology for somatic gene transfer in mammalian primary cells powered critical quality attribute (CQA) determination. Free from viral vectors, the IVT RNA method is efficient, episomal, multiplex-capable, transient but stable for sustained expression of the transgene.

Further, this Example describes an optimized approach to the mRNA based expression of opsins, or optogenetic proteins, in cardiomyocytes as a means of promoting light-based differentiation or maturation of the cells. Opsins are light-responsive ion channels which can be used for the control of cells that exhibit or respond to electrical activity. The current use of opsins for cardiac applications is limited by the vectors used for expression. The primary expression vector for opsins includes adeno-associated virus (AAV) or adenoviral vectors. This results in limitations inherent to the individual virus used—this can include permanence of expression, the induction of a strong innate immune response, and possible integration into the host genome. An mRNA expression vector allows transient expression of an opsin with fewer safety risks, especially if the cells may be transplanted into humans at a later date. Examples of opsins commonly used include the excitatory opsin, Channel-rhodopsin II (ChR2), activated with pulsed blue light, and the inhibitory opsin JAWS, activated by constant orange or red light.

For proper characterization of cells transfected with opsins, in addition to protein expression, another metric, function, can assist in properly optimizing the use of mRNA as an expression vector. Functional characterization can be performed with the use of a multi-electrode array (MEA). An MEA device uses electrodes to measure the field potential of nearby cells. Changes in field potential in cardiomyocytes can be detected in an amplitude and location-specific manner depending on the pickup electrode. The MEA device used, a Maestro from Axion Biosystems, allows the simultaneous measurement and light excitation of 16 electrodes per well in a 48 well plate. By employing primary ventricular cardiomyocytes from neonatal rats (NRVMs), functional expression of an opsin such as ChR2 can be measured as a synchronization event between electrical activity and pulsed light excitation from the LEDs embedded in the Axion Lumos device.

Derive Human Cardiomyocytes of Atrial, Ventricular and Nodal Subtypes from Two hiPS Lines in 2D and 3D Culture Conditions.

The inventors have developed a small molecule-based expertise for differentiating specific lineages of cardiac myocytes: namely, ventricular (hiPSC-VMs), atrial (AMs) and nodal pacemaker cells (PMs). The AM and VM cardiomyocytes were derived as 2D cell sheets reflecting their myocardial layers, while the PM cardiomyocytes were generated in 3D spheroids mimicking the nodal architecture of the pacemaker cells.

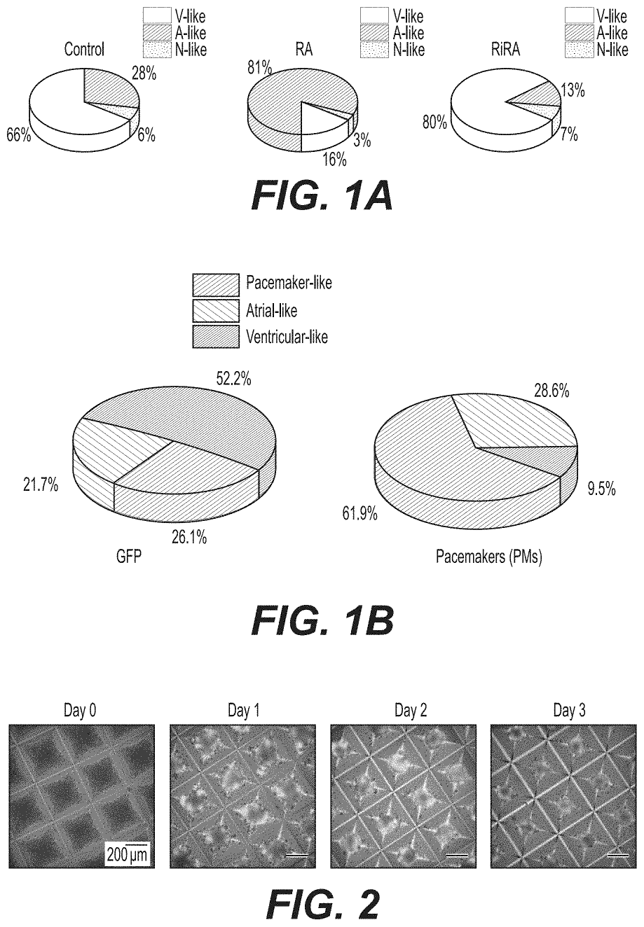

The inventors discovered that regulation of retinoic acid signal can differentially derive VMs or AMs from hiPSCs and hESCs. Furthermore, activation of canonical Wnt pathways often preferentially led to enrichment of cardiac pacemaker cell populations (FIGS. 1A-1B). These small molecule-based approaches to differentiating specific lineages of cardiac myocytes described herein are novel and simple, and are generalizable to multiple hiPSC/hESC lines.

The de novo AMs and VMs were derived as 2D cell sheets, while hiPSC-PMs were derived as 3D spheroids for downstream functional assays. The inventors have routinely derived small-scale, hiPSC-AM and -VMs as cell sheets and hiPSC-PMs as spheroids (FIG. 2). The inventors have determined that spheroids consisting of >2000 cardiomyocytes began to necrotize at the core of the spheroids over extended (>2 wks) culture period. In addition to the monolayers, the stem cell-derived human cardiac tissues were employed for functional characterization of ventricular, atrial and pacemaker phenotypes.

Upon electrical excitation, a cardiac myocyte's membrane potential undergoes nonlinear depolarization followed by repolarization to its resting state. Measurement of this action potential by a patch-clamp technique is commonly used to determine a cardiac myocyte's subtype. However, this technique is low-throughput and not feasible for screening de novo cardiac myocytes' subtypes. In contrast, optical recordings, such as those described herein, can output the membrane potential change at multi-cellular tissues as well as at a single-myocyte level.

Develop and Synthesize IVT, Modified mRNAs Enabling Optogenetic Measurements of the Principal Features of Cardiomyocytes.

The inventors have developed IVT mRNAs for cardiac myocyte-specific, functional readouts of the action potential, intracellular Ca2+ handling and contractility. The IVT optogenetic mRNAs were designed to exhibit distinct spectral properties so as to enable multiplexing of all three measurements from the same populations of the myocytes. The synthesized mRNAs can be modified to have sustained expression of the transgenes for >2 days, which is sufficient to outlast the time needed for functional characterization of the hiPSC-VMs, -AMs or -PMs.

Real-time, 6-week longitudinal analytics were performed to measure electrical and contractile functions of the de novo cardiac myocytes. The iPSC cardiomyocytes were transfected with the IVT mRNAs at three time points: at 2, 4 and 6 weeks after initial differentiation. Action potential profiles and Ca2+ transient dynamics served as the cardiac myocytes' electrophysiological readouts. Parallel measurements of contractility in the same cells by fluorescent cTNT reflected the degree of force generated by the myocytes. To validate that the platform does not interfere with the natural course of differentiation, the inventors examined gene expression profiles of the iPSC cardiomyocytes ±IVT mRNA transfection at weeks 3, 5 and 7.

IVT mRNA was produced from custom DNA templates that contain an optimized 5′-UTR (untranslated region), custom codon optimized open reading frames (ORF), as well as cell type specific 3′UTRs. Translational efficiency and mRNA stability were achieved by 5′ capping with Cap 1 enzyme with >95% efficiency, and 3′ polyadenylation with a tail >200 nucleotides (nts). The host immune response with interferon and pro-inflammatory cytokine production was mitigated by the use of (1) reverse phase HPLC to remove unwanted dsRNA, (2) codon optimization, and (3) modified nucleotides. The inventors have successfully identified a successful modification as described herein. The inventors have also tested an array of delivery systems and have determined that a modified polyethyleneimine (PEI) formulation can be optimal.

Human iPS cell-derived cardiomyocytes were seeded into 48 well Axion MEA plates and cultured for 2 weeks until completely synchronized electrical activity (beat rate) was observed. Cells were transfected with ChR2 mRNA, JAWs mRNA, delivery vehicle only, or a mixture of both mRNAs using a modified PEI derivative (Lypocalyx Gmbh). Transfected cells were assayed using the Axion Maestro MEA and Lumos light excitation device at 6 hours and 24 hours post transfection. Assays were comprised of electrical readout (16 electrodes per well) with either no stimulus, blue light stimulus (drives firing in ChR2-transfected cells), orange light stimulus (inhibits firing in JAWs-transfected cells), or blue light impulse stimulus with a period of constant orange light.

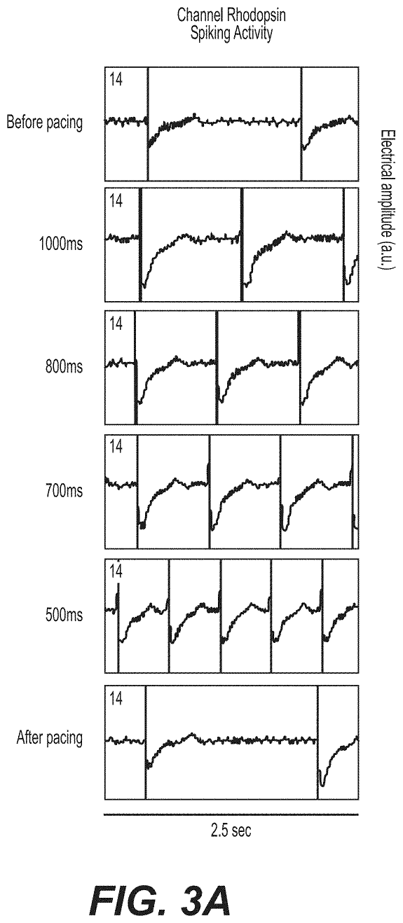

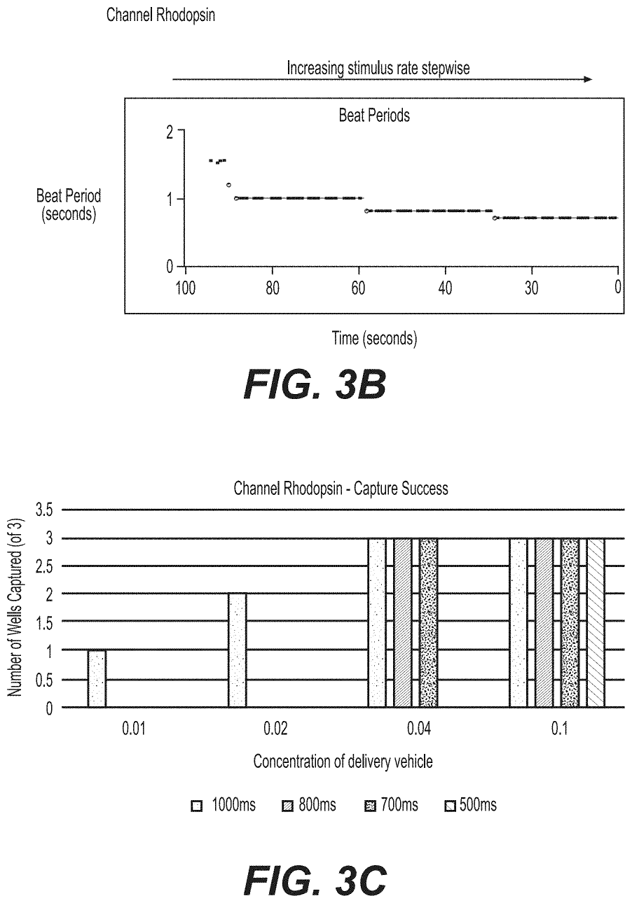

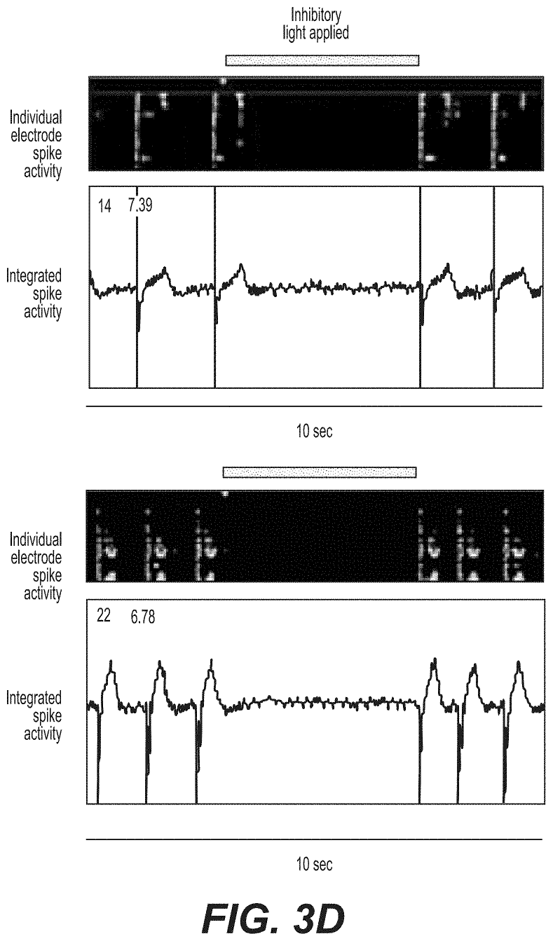

As a preliminary step, the inventors determined the optimal amount of delivery vehicle to transfect cells for adequate function of the expressed mRNA, defined as complete control of beat rate with ChR2 and conversely complete inhibition of firing with JAWs. Changes in beat rate were observed in cardiomyocytes at 6 hours post-transfection, though complete capture (synchronization with firing or complete inhibition) were not observed until 24 hours with the second highest and highest amounts of delivery vehicle (FIG. 3A-3D). Delivery vehicle alone had no effect on native cell beat rates, thus it is likely that higher amounts of protein can be generated if opsin function is required more rapidly. Increasing rates of blue light spike stimulus applied to ChR2 mRNA-transfected cells resulted in increasing beat rates with complete synchronization to 500 ms of beat-to-beat interval (FIG. 3A-3B). Attempts to drive beat rates faster resulted in aliasing to lower beat rates, implying a minimum reset time for continuous firing. Using 0.1 ul of delivery vehicle, all three wells (100%) were paced via light (FIG. 3C). JAWS mRNA-transfected cells inhibited beating completely during the period of 10 seconds when orange light was continuously applied (FIG. 3D). The same functions were observed in cardiomyocytes transfected with both JAWS and ChR2 mRNA.

The inventors have shown function and time-dependence of expression of mRNA encoding opsins in cardiomyocytes. Compared to viral opsin expression vectors, functional expression is achievable in one day as opposed to weeks. Following these results, the inventors generated IVT mRNA for ChRh2 (optogenetic electrical excitation), ArchD95H (red-shifted voltage-sensor protein), GcaMP6f (green-shifted Ca2+ sensor protein), and cTNT-E2Crimson (far red-shifted cardiac contractility protein).

Real-time, 6-week longitudinal analytics were performed to measure electrical and contractile functions of the de novo cardiac myocytes. The inventors next evaluated two primary functional outputs of cardiomyocytes in a non-invasive, all-optical manner. The iPSC cardiomyocytes were transfected with the IVT mRNAs at three time points: at 2, 4 and 6 weeks after initial differentiation. Action potential profiles and Ca2+ transient dynamics served as the cardiac myocytes' electrophysiological hallmarks. A high-sensitivity, high-resolution ORCA4.0 sCMOS camera paired with LEICA DMi8 optics was used to collect the voltage signal and Ca2+ signal at frame rates >700 fps.

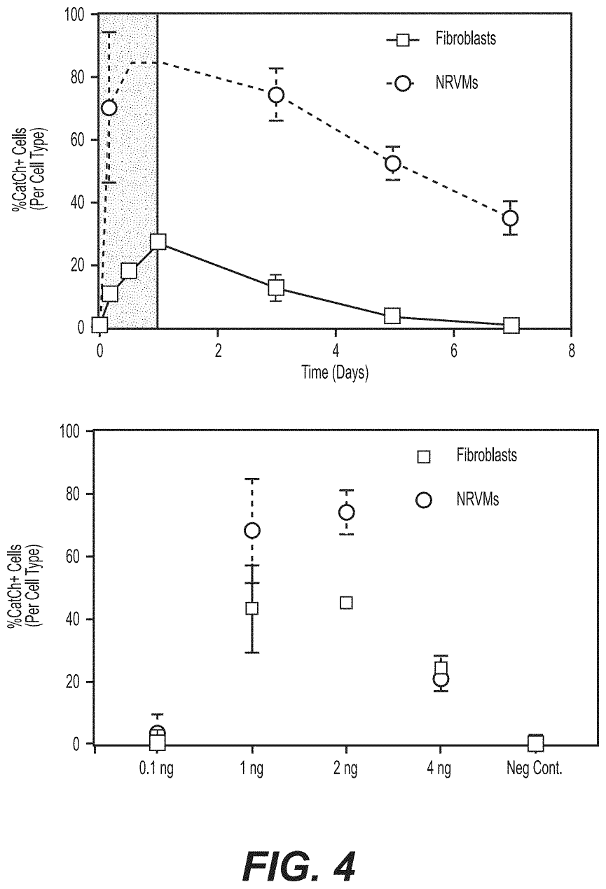

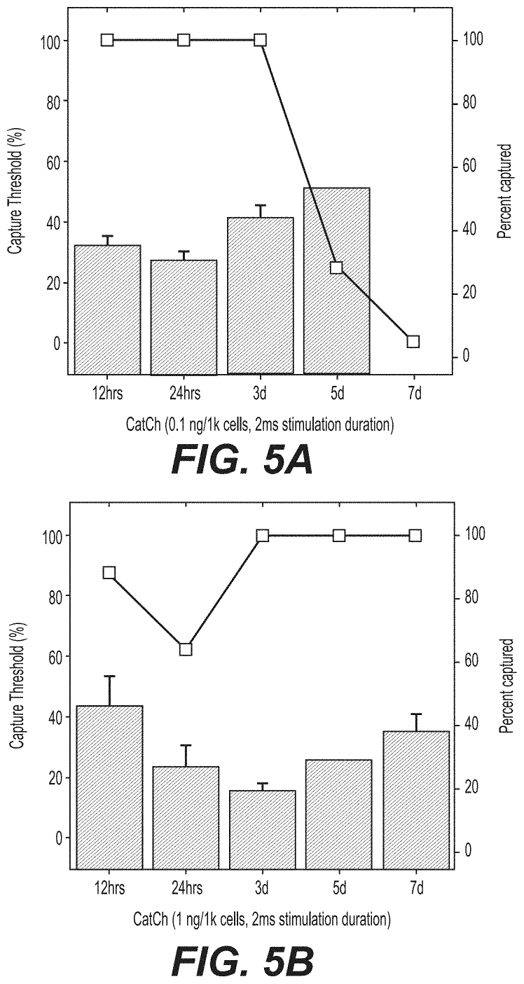

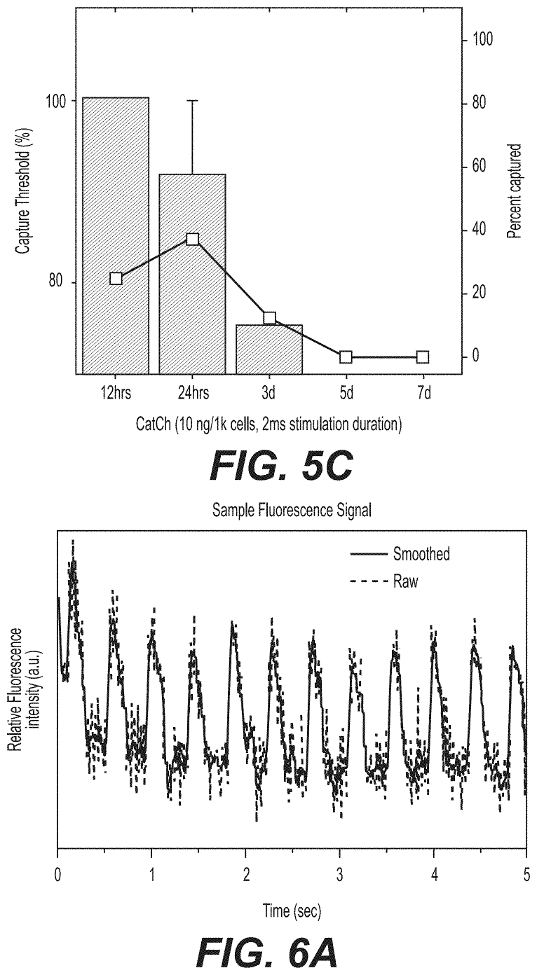

As a first step, the inventors expressed CatCh in neonatal rat ventricular cardiomyocytes (NRVMs) using IVT mRNA. Transfection conditions were varied using GFP mRNA. After identifying the optimal vehicle for transfection (Viromer Red), the expression of the protein of interest, CatCh-V5, was assessed after a 24 hour pulse of mRNA. From the imaging time course, the inventors saw that strong CatCh-V5 expression was achieved in NRVMS, identified by expression of alpha-SA (a marker of cardiomyocytes). CatCh-V5 localized to the membrane of transfected cells roughly 12 hours after transfection. Expression peaked at 24 hours, dropping off significantly by day 5. Flow cytometry, shown in FIG. 4, showed efficient transfection of NRVMs over fibroblasts in culture, and the time course profile matched what was seen in the time course images. The inventors next evaluated the function of transfected NRVMs. CatCh is an opsin, which induces a membrane depolarization in response to blue light stimulation. CatCh transfected NRVMs were exposed to a range of optical intensities (0 to 100% power) using 475 nm blue LED light. At each light intensity, the inventors scanned a range of stimulation durations (1 to 15 milliseconds). A train of monophasic square waves at a frequency of 2 Hz for a total of 30 seconds was utilized. The inventors compared the capture rate of CatCh versus Channelrhodopsin 2 (ChR2), a commonly used opsin. CatCh demonstrated superior sensitivity, resulting in a higher percentage of captured cells at lower light intensity and a shorter pulse duration. Cells were then subjected to blue light stimulation and analyzed for capture at different transfection amounts and timepoints. FIG. 5A-5C shows the results of this experiment, which indicated that 1 ng/1,000 cells was the optimal RNA dose to achieve reliable pacing over time, and that days 1-3 post-transfection allowed for the highest capture rates at the lowest stimulation thresholds.

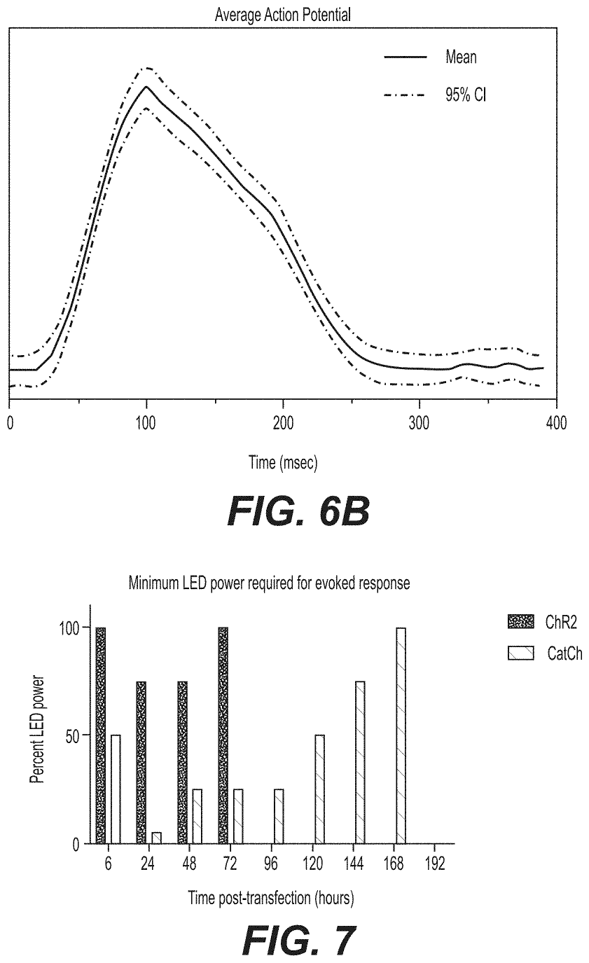

The inventors next sought to demonstrate the ability to use light to characterize the electrical properties of the NRVMs. FlicR is a voltage indicator protein that fluoresces at different intensities depending on the electrical potential of the cell membrane. By transfecting cells with mRNA encoding FlicR, the characteristics of their action potentials can be assessed on a large number of cells using microscopy. The inventors transfected NRVMs with mRNA encoding this protein and imaged them on a swept field microscope in live culture. A series of images were obtained at 100 Hz and brightness over time measurements were performed in a region of interest per cell. The signal was processed using a custom MATLAB script. FIG. 6A-6B show a sample plot of a single cell's action potential over time (FIG. 6A) and the average action potential captured for that cell (FIG. 6B). An electric pacing system can also be employed to pace the entire cell monolayer at a specific rate while imaging for FlicR intensity. The FlicR mRNA system can be used to characterize the electrical phenotypes of cardiomyocytes derived from human iPS cells to attain their critical quality attributes in an all-optogenetic manner.

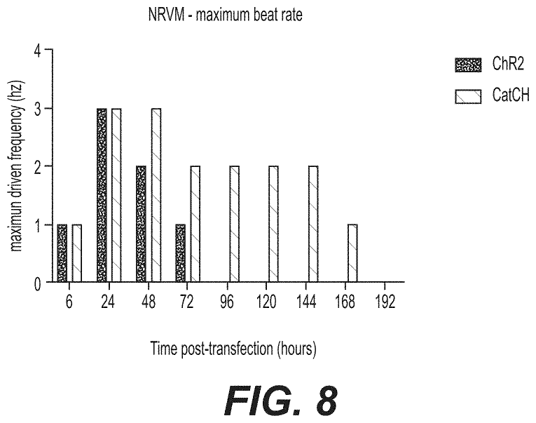

Pacing cardiac myocytes allows the screening of new drugs in a controlled environment, where the effect on beat frequency and waveforms can be measured against predicted values in the presence/absence of drugs. The inventors transfected neonatal rat ventricular myocytes (NRVMs) with mRNA encoding ChR2 and a modified ChR2 with a single amino acid substitution, CatCH. NRVMs were plated on MEA plates and the next day were transfected with ChR2 or CatCH mRNA at varying concentrations in triplicate wells using Viromer Red (Lipocalyx). CatCH-transfected cells exhibited electrical responses to 15 times less light than ChR2 at 24 hours post-transfection (FIG. 7). Furthermore, when interrogated with excitation rates of 1 hz, 2 hz, and 3 hz, CatCH transfected cells were able to be driven at higher beat rates (3 hz) up to 72 hours post-transfection (FIG. 8) compared to ChR2 transfected cells. As the amount of mRNA and structure of the opsin are very similar, this difference can be inferred as due to enhanced opsin sensitivity—fewer opsins per cell were necessary to drive cardiac action potentials. When opsin expression was low, such as at timepoints several days following transfection, cardiac cells could only be driven at the slower rate of 1 hz successfully.

Thus, cardiac myocytes were transfected efficiently with mRNA, with expression duration up to 144 hours post-transfection and higher light sensitivity based on MEA measurements. CatCH performed with much higher sensitivity than ChR2, at a much lower amount of mRNA (125 ng of CatCH versus 1000 ng of ChR2).

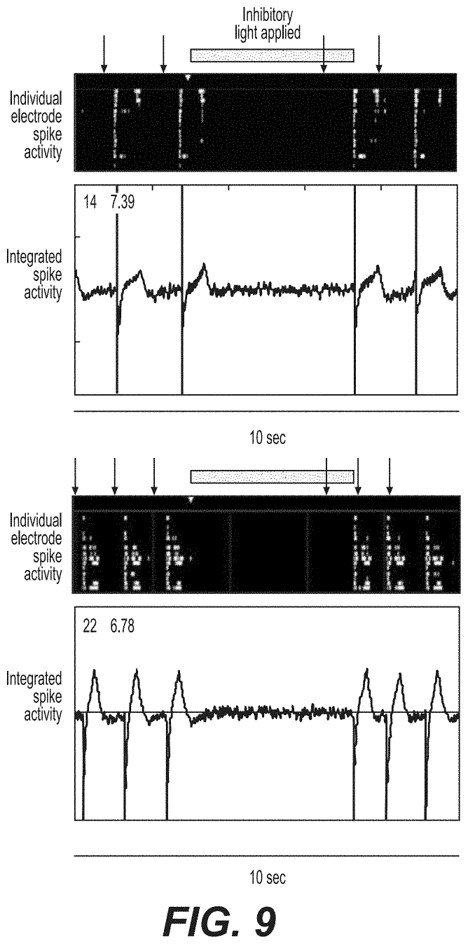

The inventors next tested expression and function of ChR2 and JAWS (inhibitory opsin) in iPS-derived cardiomyocytes. Here, 500 ng each of ChR2 and JAWS mRNA were delivered to iPS-derived cardiomyocytes in an MEA plate (FIG. 9). The next day, cells were paced using pulsed blue light stimulation (arrows). At the same time, orange light was used for several seconds (bars) to prevent cardiomyocytes from beating. This showed function of both opsins in a dual transfection.

### Example 3. Development of a mRNA Encoding a Differentiation Factor and Ability to Cause Cardiomyocyte Differentiation in an Animal Model of Heart Block

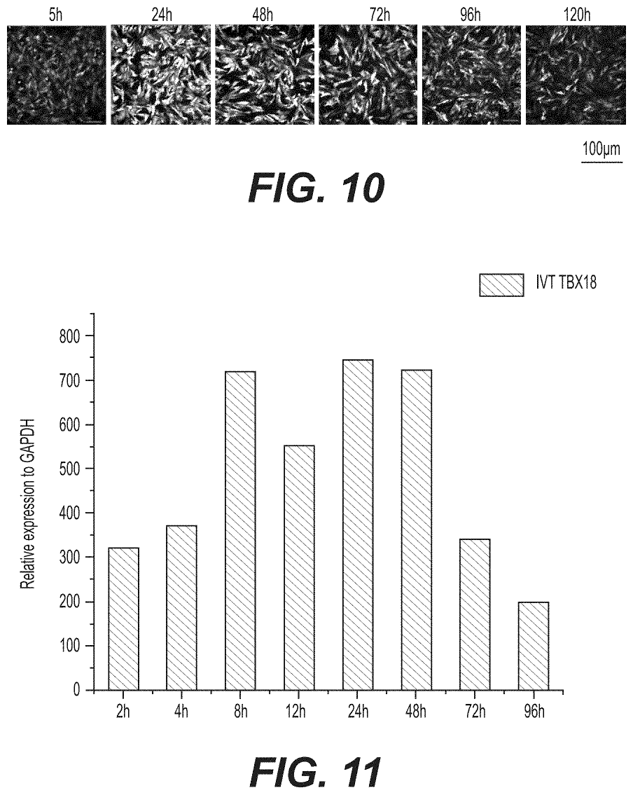

Freshly-isolated neonatal rat ventricular cardiomyocytes were transfected with IVT mRNA for green fluorescent protein (GFP). Somatic gene transfer was performed by adding 333 ng of GFP IVT mRNA with a transfection reagent, ViroMer Red, to a well in a 96-well plate seeded with the neonatal rat ventricular cardiomyocytes. To examine successful gene transfer and protein expression of the transgene, fluorescence emitted by GFP was imaged at the indicated times after the transfection. Greater than 98% of cardiomyocytes were transfected with the IVT mRNA, exhibited robust expression of the exogenous protein, and the transgene expression was transient evidenced by the weaker GFP signal over time (FIG. 10).

The inventors next studied the ability of TBX18 IVT mRNA to transfect cardiac myocytes and found that this mRNA successfully and efficiently entered cardiac myocytes in a transient manner (FIG. 11). Freshly-isolated neonatal rat ventricular cardiomyocytes were transfected with IVT mRNA for human TBX18 gene. Somatic gene transfer was performed by adding 333 ng of TBX18 IVT mRNA with a transfection reagent, ViroMer Red, to a well in a 96-well plate seeded with the neonatal rat ventricular cardiomyocytes. TBX18 IVT mRNA that successfully entered the cardiomyocytes were quantitated by quantitative real-time PCR (qPCR), and the TBX18 mRNA level was normalized to an endogenous housekeeping gene, GAPDH, over time. The data demonstrated efficient entry of TBX18 IVT mRNA, and the transient nature of the delivered mRNA.

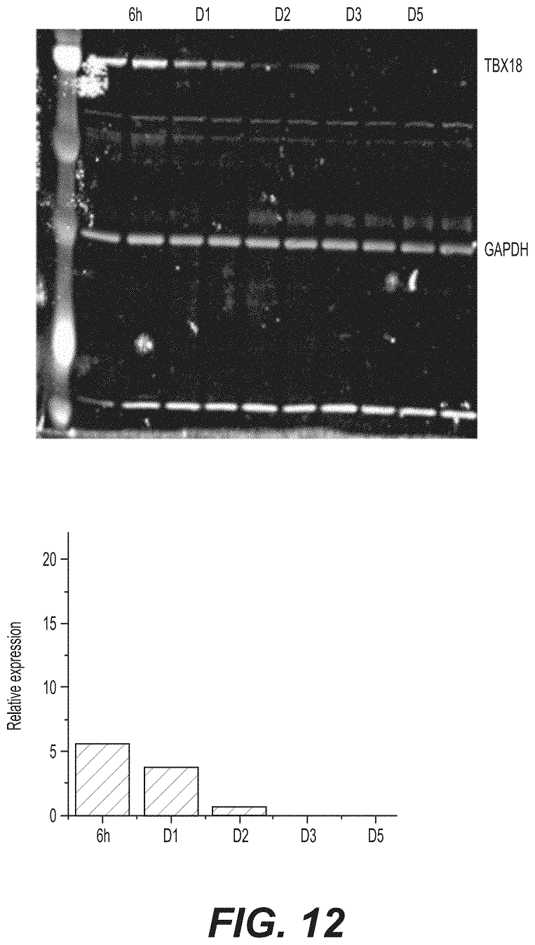

Expression of the TBX18 protein was then monitored, and it was found that TBX18 protein was efficiently and transiently expressed in cardiac myocytes (FIG. 12). Freshly-isolated neonatal rat ventricular cardiomyocytes were transfected with IVT mRNA for human TBX18 gene. Somatic gene transfer was performed by adding 333 ng of TBX18 IVT mRNA with a transfection reagent, ViroMer Red, to a well in a 96-well plate seeded with the neonatal rat ventricular cardiomyocytes. TBX18 protein expression from the transfected IVT mRNA in the cardiomyocytes were quantitated by specific antibodies to TBX18 on immunoblots and normalized to an endogenous housekeeping protein, GAPDH. The data demonstrated efficient and immediate TBX18 protein expression, in less than 6 hours post-transfection. The TBX18 protein expression was also transient, in line with the expression kinetics of the delivered mRNA. This transient expression of TBX18 closely mimicked its native expression during embryonic development, and is ideal for the intended purpose as a reprogramming agent to convert ordinary heart muscle cells to specialized cardiac pacemaker cells. In FIG. 12, D denotes days after transfection, and each time point consists of two technical replicates.

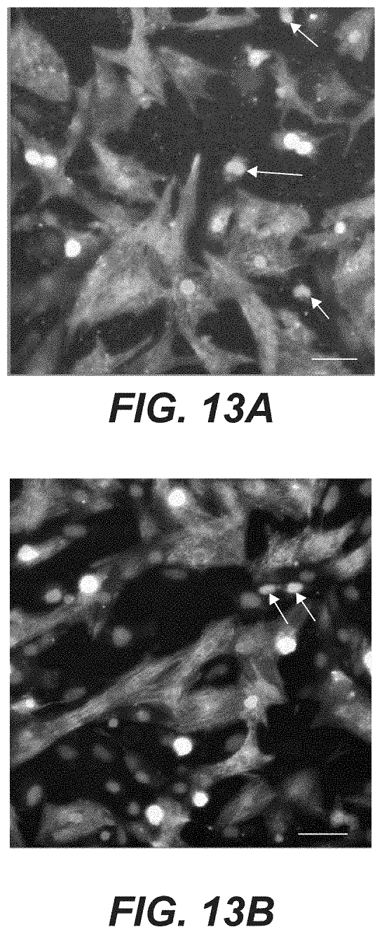

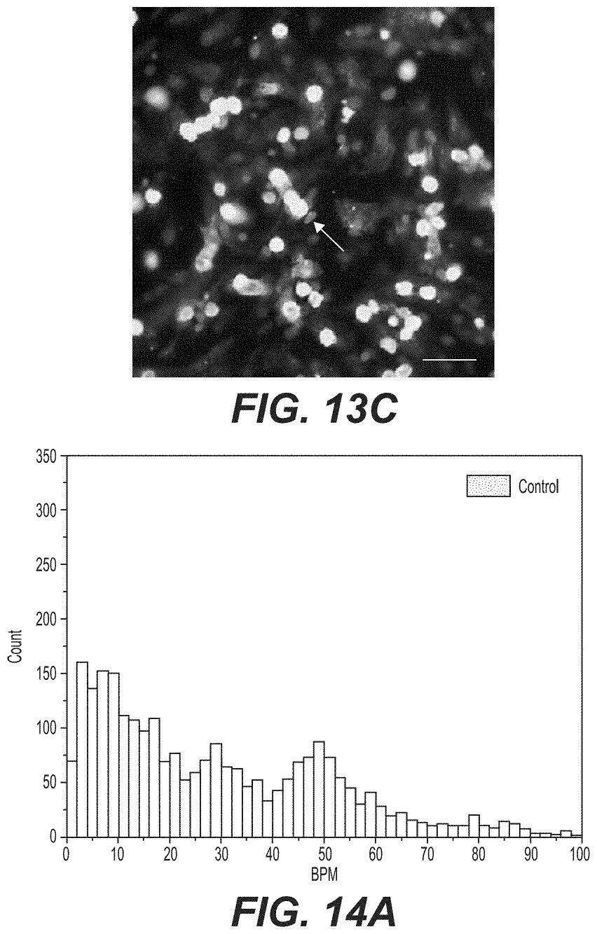

The inventors next determined the localization of TBX18 using fluorescence microscopy and found that TBX18 did correctly localize to the cell nuclei (FIG. 13A-13C). TBX18 is a transcription factor, and thus should be targeted to the nuclei upon proper translation from mRNA. Freshly-isolated neonatal rat ventricular cardiomyocytes were transfected with IVT mRNA for human TBX18 gene. TBX18 mRNA was designed with a tag peptide (DYKDDDDK (SEQ ID NO:9, also known as a “FLAG” epitope) fused to the C-terminal end of TBX18 for subcellular identification of TBX18 with an antibody against FLAG. Sarcomeric alpha-actinin (α-SA) and DAPI label cardiomyocytes and all nuclei, respectively. Arrows (co-localization of FLAG and DAPI) indicate nuclear localization of TBX18 protein. Three transfection conditions were tested in a 24-well plate, and immunostaining was performed at 24 hrs post-transfection.

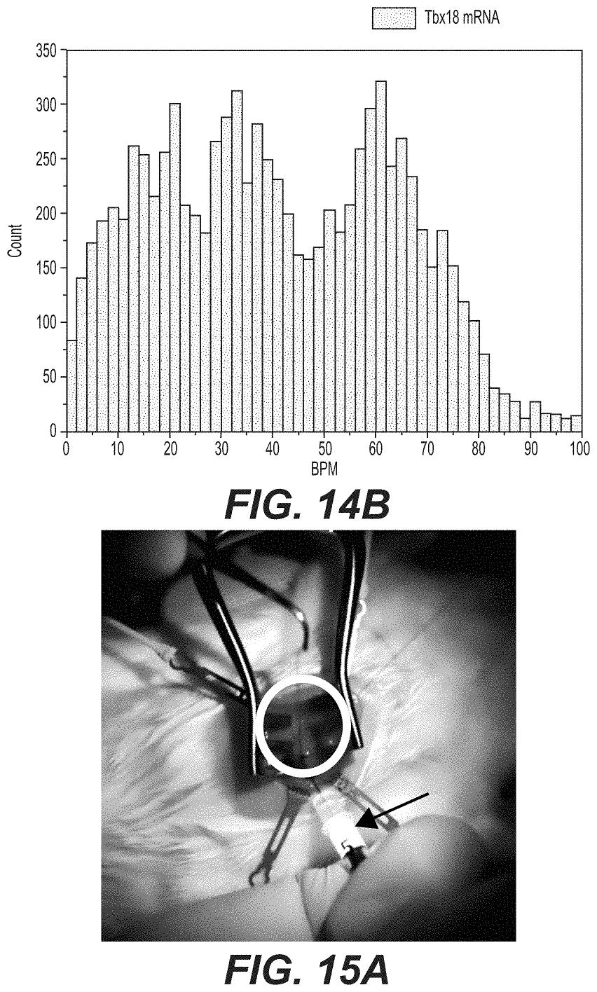

The inventors then determined the effect of expressing TBX18 mRNA in cardiac myocytes, and found that TBX18 IVT mRNA gene transfer converted ordinary heart muscle cells to de novo pacemaker cells (FIG. 14A-14B). Spontaneous electrical activity from neonatal rat ventricular myocytes were recorded on a non-invasive, multi-electrode array platform. Control cardiomyocytes (14A) exhibited infrequent and low rate activity. Gene transfer of TBX18 IVT mRNA (14B) elicited highly active, high rate pacemaker activity from the cardiomyocytes. The number of electrical activity (Count) was plotted as a function of spontaneous beating rates (in beats per minute, BPM) of the cardiomyocytes.

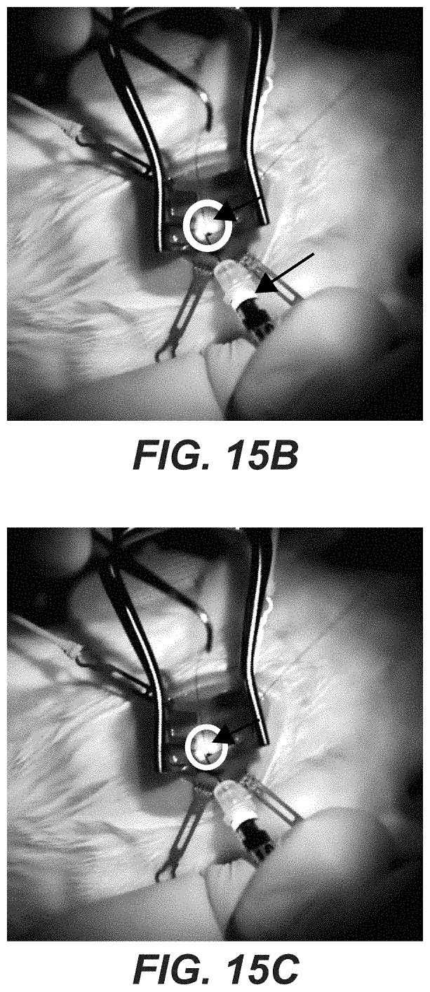

Next, the inventors directly injected a test IVT mRNA into myocardial tissue (FIG. 15A-15C). GFP IVT mRNA was conjugated with an infra-red moiety in order to visualize the mRNA before (15A) during (15B) and after (15C) the injection of the biologic to the rat heart. The mRNA with a bright glow, black arrows, was directly injected into the myocardium in an open chest, survival surgery. The bolus injection remained focal to the injection site in the heart (white oval).

The inventors next studied the effects of administering TBX18 mRNA to a rat model of a complete atrioventricular block. It was found that focal intramyocardial injection of in vitro-transcribed TBX18 mRNA created in vivo biological cardiac pacing in this rat model. In this study, the inventors created a complete atrioventricular block (CAVB) model in rats and monitored the rats for one week to confirm stable and persistent CAVB. Next, TBX18 mRNA was injected into the ventricular apex of each rat and a telemetry device was implanted. Some rats received a high dose of TBX18 mRNA, which was 300 μg TBX mRNA with 205 μl RNA free PBS. The inventors continuously monitored and recorded data from the telemetry device including electrocardiograms for 21 days. The inventors performed 3-lead surface ECGs on anesthetized animals on days 7, 14 and 21 with an isoproterenol challenge, and harvested the heart on day 21.

The inventors determined if there was antegrade conduction (meaning that the direction of cardiac conduction occurred from the SA node, AV node and down toward the apex of the heart) or retrograde conduction (meaning that the direction of cardiac conduction occurred from the site of TBX18 delivery which was the apex of the left ventricle). In order to determine this, the inventors determined a baseline ECG under anesthesia before TBX18 mRNA injection by recording three-lead surface ECGs for ten minutes while the rat was asleep under anesthesia. Recordings confirmed the presence of AV conduction block with multiple P waves between each QRS complex, inconsistent P-R intervals, and a relatively low heart rate of 100 bpm, compared to a healthy rate of over 300 bpm for rats. Cardiac axis mapping of ECG recordings showed an average conduction vector of 77 degrees, which aligned with a normal anterograde conduction pathway (atrial to ventricles).

One week after injection of TBX18 mRNA, 3-lead surface ECGs were again recorded, and there was an increase in average heart rate of 120 bpm. Additionally, observed AV block showed a 2:1 conduction between atrial and ventricles, indicating an improvement in severity of heart block. Cardiac axis mapping of ECG recordings indicated a small shift in the conduction vector to 49 degrees, still qualifying as normal anterograde conduction. These values were compared to rats who were given isoproterenol, an analog for epinephrine. In rats who received isoproterenol, the three-lead surface ECGs performed at one week after injection of TBX18 mRNA showed an increase in heart rate to 229 bpm while still maintaining features of AV conduction block (inconsistent P-R interval). The polarity and shape of the QRS complex changed to negative and wide, respectively. This demonstrated a retrograde conduction, originating near the apex of the heart which was the injection site of TBX18 mRNA.

Three weeks after injection of TBX18 mRNA, 3-lead surface ECG recordings showed pre-ventricular contractions (PVCs) as well as slow junctional escape rhythm. The average heart rate was calculated to be 137 bpm. Cardiac axis mapping of ECG recordings displayed two conduction vector phenotypes, indicative of competing conduction sources. Normal QRS complexes had anterograde conduction vectors at 48 degrees, while PVCs had retrograde conduction vectors at −116 degrees. These values were again compared to rats who were given isoproterenol. In rats who received isoproterenol, the three-lead surface ECGs performed at three weeks after injection of TBX18 mRNA showed that the injection of isoproterenol induced several transient QRS complexes during the ECG recordings. These transient QRS complexes produced retrograde conduction vectors of −110 degrees indicating pacing from the apex of the heart, which is the site of TBX18 mRNA injection.

ECG data was then compared with telemetry data. Before injection of TBX18 mRNA, telemetry biopotential electrodes were sutured to the chest wall and housed in the abdominal cavity of the rat. Telemetry 2-lead ECG signal was recorded wirelessly for conscious, awake rats. The day before injection of TBX18 mRNA, AV block was present in ECG recordings with an average heart rate of 120 bpm, and these values were used as the baseline.

One week after delivery of TBX18 mRNA, telemetry ECG recordings revealed retrograde QRS complexes during isoproterenol treatment. Isoproterenol elevated the heart rate to 265 bpm. Two weeks after delivery of TBX18 mRNA, telemetry ECG recordings again revealed retrograde QRS complexes during isoproterenol treatment. Isoproterenol elevated the heart rate to 206 bpm. Three weeks after delivery of TBX18 mRNA, conscious rats still displayed retrograde QRS complexes during isoproterenol treatment, and heart rate was again elevated to 260 bpm.

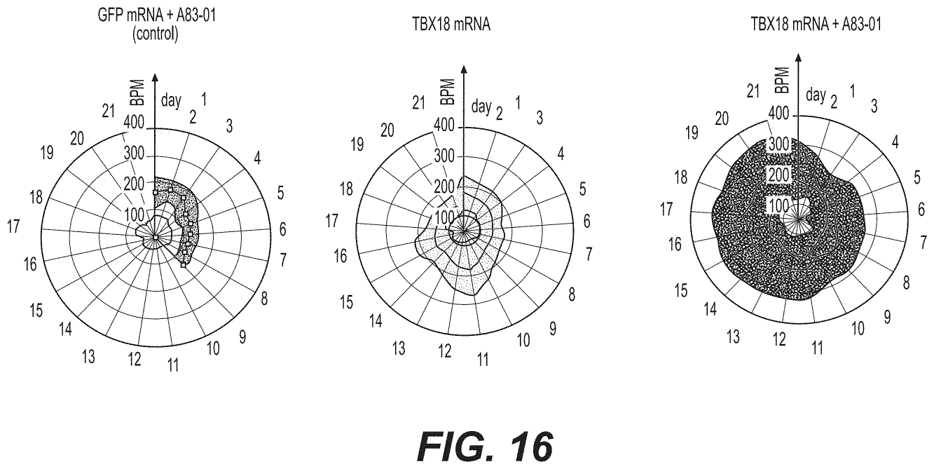

The inventors then determined the effects of including a TGF-beta small molecule inhibitor on ventricular pacing with TBX18 mRNA (FIG. 16). It was found that administering TBX18 mRNA created a complete ventricular block in a rat model of complete heart block. Administering the TGF-beta small molecule inhibitor A83-01 reversed the block.

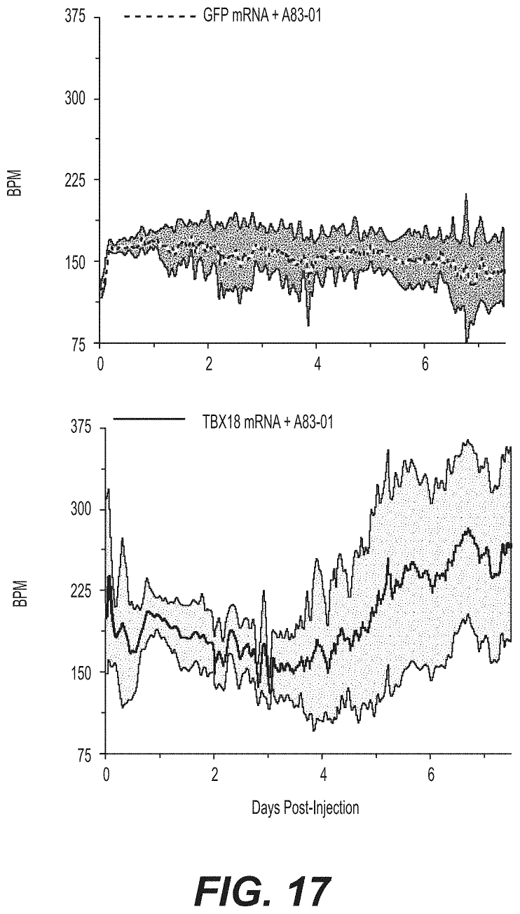

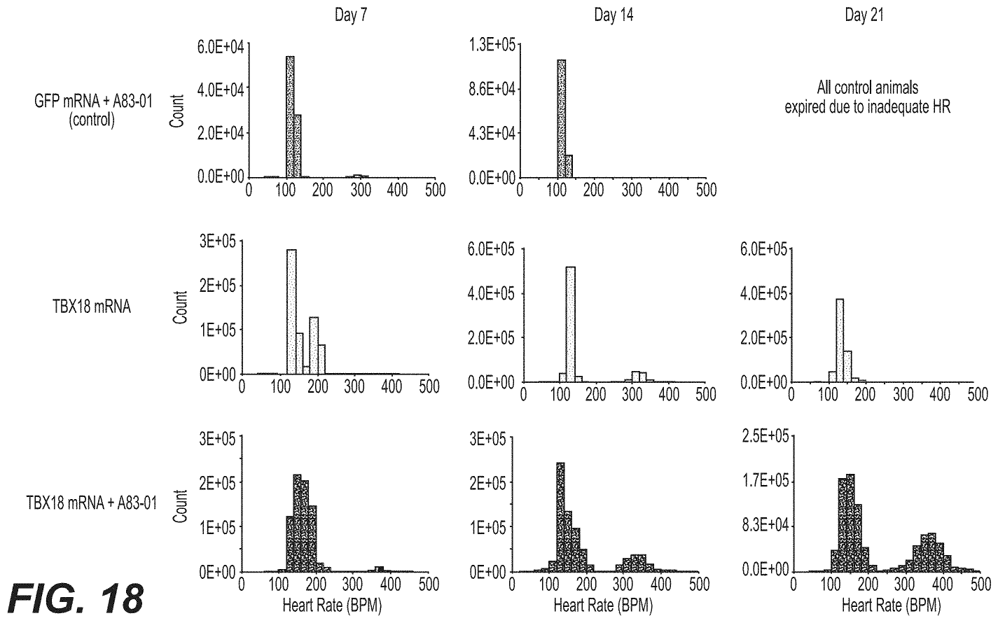

The inventors further demonstrated that TBX18 mRNA gene transfer created ventricular pacing in a rat model of complete heart block by continuous recording of the animals' heart rate (FIG. 17) and heart rate histograms performed on Days 21 after injection (FIG. 18).

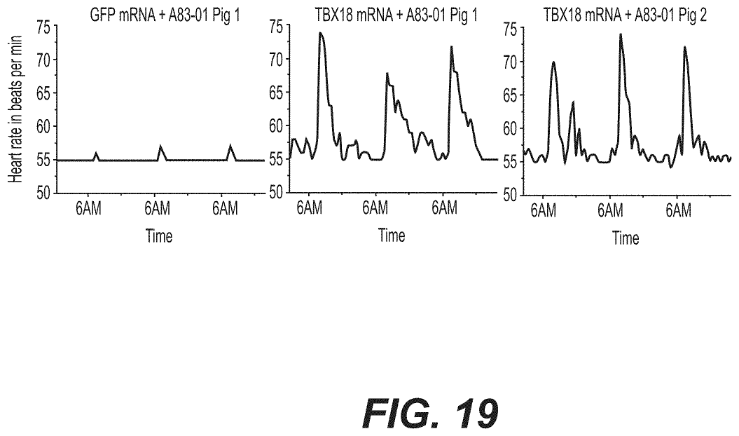

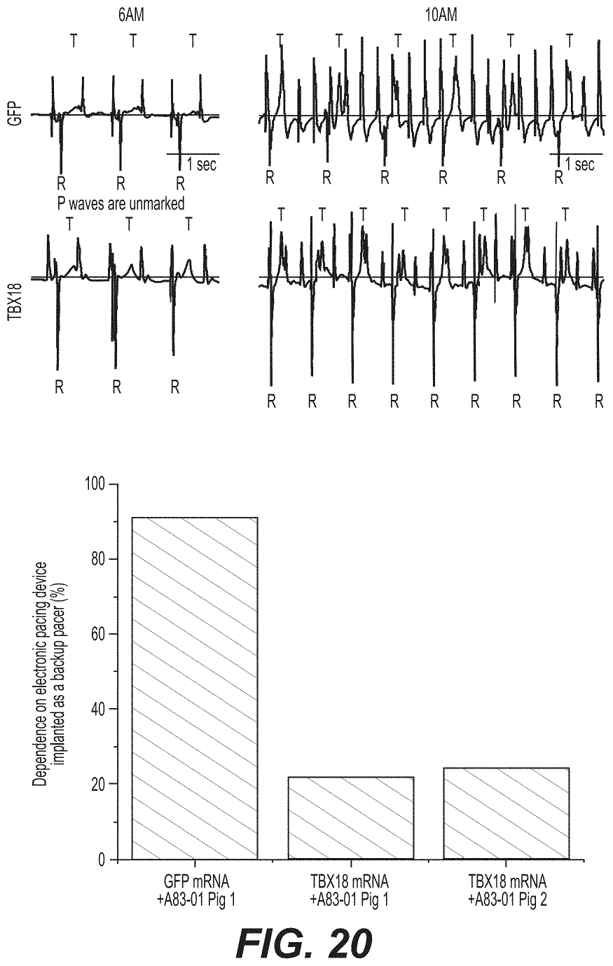

The inventors next sought to test the TBX18 mRNA in a large animal model, specifically a porcine model, of complete heart block. The inventors used a control of GFP mRNA+A83-01. On Day 1, the inventors created an intracardiac map by implanting a pacemaker and administered the TBX18 mRNA or control. Weeks 2, 4, and 6 after administration included an interrogation of the pacemaker and an isoproterenol challenge to measure the animals' maximum heart rate (max HR). In Week 6, arrythmia testing and intracardiac mapping were performed and vital organs were harvested. The inventors found that TBX18 mRNA created ventricular pacing in a porcine model within days 7-10 after delivery of the mRNA (FIG. 19). The inventors also found that TBX18 mRNA with A83-01 created ventricular pacing in a porcine model (day 10 after biologic delivery), and that TBX18 mRNA injected into the porcine model shows shorter R-R interval (faster heart rate) as the pig becomes awake in the morning. The proper diurnal response in TBX18-injected pig is in line with the average heart rate data from FIG. 18.

### LIST OF EMBODIMENTS

1. A composition comprising:

(i) at least one messenger RNA (mRNA) encoding a differentiation factor, a transcription factor, and/or a phenotype sensor; and

(ii) a delivery vehicle comprising a cationic lipid, a polyethylenimine (PEI) derivative, a polymer, a polypeptide or peptide, a nanoparticle, or a lipid-based particle.

2. The composition of embodiment 1, wherein the at least one mRNA encodes a differentiation factor selected from the group consisting of Tbx18, Tbx3, Tbx5, and SHOX2 and combinations thereof.
3. The composition of embodiment 1 or 2, wherein the at least one mRNA encodes a phenotype sensor selected from the group consisting of an opsin and a protein capable of sensing cell electrophysiology and combinations thereof.
4. The composition of embodiment 3, wherein the phenotype sensor is selected from the group consisting of Quasar, Jaws, Catch-V5, ChR2, Archer1, FlicR1, ArcD95H, GCaMP6f, and cTNT-E2Crimson and combinations thereof.
5. The composition of any of embodiments 1-4, wherein the at least one mRNA comprises at least one modified nucleotide, a cap, at least one untranslated region, a polyadenine tail, and/or at least one nucleotide mutation resulting in codon optimization.
6. The composition of any of embodiments 1-5, wherein the delivery vehicle comprises a cationic lipid comprising lipofectamine.
7. The composition of any of embodiments 1-5, wherein the delivery vehicle comprises a polyethylenimine derivative selected from the group consisting of linear polyethyleneimine derivatives.
8. The composition of any of embodiments 1-5, wherein the delivery vehicle comprises a polymer selected from the group consisting of virus-like polymers.
9. The composition of any of embodiments 1-5, wherein the delivery vehicle comprises a nanoparticle selected from the group consisting of viruses and virus-like particles.
10. The composition of any of embodiments 1-5, wherein the delivery vehicle comprises a lipid-based particle selected from the group consisting of liposomes and nanoliposomes.
11. The composition of any of embodiments 1-10, further comprising a small molecule attached to the at least one mRNA.
12. The composition of embodiment 11, wherein the small molecule is selected from the group consisting of inhibitors of innate immune sensors.
12A. The composition of any of embodiments 1-12, wherein the at least one mRNA is present in an mRNA expression vector.
12B. A host cell transfected with any of the compositions of embodiments 1-12A.
12C. The host cell of embodiment 12B, wherein the host cell is selected from the group consisting of a cardiac cell, a cardiomyocyte, a neuronal cell, a cell located within the eye, a pancreatic cell, a PSC, an IPSC, an ESC, and a PSC cardiomyocyte.
13. A method of modulating cell phenotypes comprising the steps of:

(i) formulating a composition comprising:
at least one mRNA expression vector encoding a differentiation factor, a transcription factor, and/or a phenotype sensor; and
a delivery vehicle comprising a cationic lipid, a polyethylenimine (PEI) derivative, a polymer, a polypeptide or peptide, a nanoparticle, or a lipid-based particle;

(ii) administering the composition to a target cell via transfection;

(iii) optionally detecting the phenotype of the target cell; and

(iv) optionally providing one or more phenotypically appropriate target cells to a patient to treat and/or prevent a disease.

14. The method of embodiment 13, wherein the at least one mRNA encodes a differentiation factor selected from the group consisting of Tbx18, Tbx3, Tbx5, and SHOX2 and combinations thereof.
15. The method of embodiments 13 or 14, wherein the at least one mRNA encodes a phenotype sensor selected from the group consisting of an opsin and a protein capable of sensing cell electrophysiology and combinations thereof.
16. The method of embodiment 15, wherein the phenotype sensor is selected from the group consisting of Quasar, Jaws, Catch-V5, ChR2, Archer1, FlicR1, ArcD95H, GCaMP6f, and cTNT-E2Crimson and combinations thereof.
17. The method of any of embodiments 13-16, wherein the at least one mRNA comprises at least one modified nucleotide, a cap, at least one untranslated region, a polyadenine tail, and/or at least one nucleotide mutation resulting in codon optimization.
18. The method of any of embodiments 13-17, wherein the delivery vehicle comprises a cationic lipid comprising lipofectamine.
19. The method of any of embodiments 13-17, wherein the delivery vehicle comprises a polyethylenimine derivative selected from the group consisting of linear PEI derivatives.
20. The method of any of embodiments 13-17, wherein the delivery vehicle comprises a polymer selected from the group consisting of virus-like polymers.
21. The method of any of embodiments 13-17, wherein the delivery vehicle comprises a nanoparticle selected from the group consisting of viruses and virus-like particles.
22. The method of any of embodiments 13-17, wherein the delivery vehicle comprises a lipid-based particle selected from the group consisting of liposomes and nanoliposomes.
23. The method of any of embodiments 13-22, further comprising a small molecule tethered to the at least one mRNA.
24. The method of embodiments 23, wherein the small molecule is selected from the group consisting of inhibitors of innate immune sensors.
25. The method of any of embodiments 13-24, wherein the target cell is selected from the group consisting of a cardiac cell, a cardiomyocyte, a neuronal cell, a cell located within the eye, a pancreatic cell, a PSC, an IPSC, an ESC, and a PSC cardiomyocyte.
26. The method of embodiment 25, wherein the target cell is a cardiac cell, a cardiomyocyte, a PSC, or a PSC cardiomyocyte, and wherein the phenotype of the target cell has been modified to cause the target cell to differentiate into an atrial myocyte, a ventricular myocyte, and/or a cardiac pacemaker cell.
27. The method of embodiment 25, wherein the differentiated cardiac pacemaker cell is provided to a subject in need thereof.
28. The method of embodiment 27, wherein the subject has an arrythmia, a cardiac pacing disorder, atrioventricular block, or sick sinus syndrome.
29. A method of treating and/or preventing a cardiac disorder in a subject in need thereof comprising the steps of:

(i) formulating a composition comprising:
at least one messenger RNA (mRNA) encoding a differentiation factor, a transcription factor, and/or a phenotype sensor; and
a delivery vehicle comprising a cationic lipid, a polyethylenimine (PEI) derivative, a polymer, a polypeptide or peptide, a nanoparticle, or a lipid-based particle;

(ii) administering the composition to a target cell selected from the group consisting of a cardiac cell, a cardiomyocyte, a PSC, an IPSC, an ESC, and a PSC cardiomyocyte via transfection;

(iii) optionally detecting the phenotype of the transfected target cell by detecting the activity of the phenotype sensor; and

(iv) re-implanting the transfected target cell in the subject, where the subject optionally has an electric pacemaker device.

30. The method of embodiment 29, wherein the cardiac disorder is selected from the group consisting of atrioventricular block, sick sinus syndrome, and other arrhythmias which typically require the implantation of an electronic pacemaker device.
31. The method of embodiments 29 or 30, wherein the step of detecting the phenotype of the transfected cardiac cells comprises one or more of performing multi-electrode array (MEA) analysis on the cell, measuring action potential profiles and/or Ca2+ transient dynamics, and/or measuring fluorescent cTNT.
32. The method of any of embodiments 29-31, wherein the at least one mRNA encodes a differentiation factor selected from the group consisting of Tbx18, Tbx3, Tbx5, and SHOX2 and combinations thereof.
33. The method of any of embodiments 29-32, wherein the at least one mRNA encodes a phenotype sensor selected from the group consisting of an opsin and a protein capable of sensing cell electrophysiology and combinations thereof.
34. The method of embodiment 33, wherein the phenotype sensor is selected from the group consisting of Quasar, Jaws, Catch-V5, ChR2, Archer1, FlicR1, ArcD95H, GCaMP6f, and cTNT-E2Crimson and combinations thereof.
35. The method of any of embodiments 29-34, wherein the at least one mRNA comprises at least one modified nucleotide, a cap, at least one untranslated region, a polyadenine tail, and/or at least one nucleotide mutation resulting in codon optimization.
36. The method of any of embodiments 29-35, wherein the delivery vehicle comprises a cationic lipid comprising a lipofectamine.
37. The method of any of embodiments 29-35, wherein the delivery vehicle comprises a polyethylenimine derivative selected from the group consisting of linear PEI derivatives.
38. The method of any of embodiments 29-35, wherein the delivery vehicle comprises a polymer selected from the group consisting of virus-like polymers.
39. The method of any of embodiments 29-35, wherein the delivery vehicle comprises a nanoparticle selected from the group consisting of viruses and virus-like polymers.
40. The method of any of embodiments 29-35, wherein the delivery vehicle comprises a lipid-based particle selected from the group consisting of liposomes and nanoliposomes.
41. The method of any of embodiments 29-35, further comprising a small molecule tethered to the at least one mRNA.
42. The method of embodiment 41, wherein the small molecule is selected from the group consisting of inhibitors of innate immune sensors.
43. A method of determining a cell phenotype comprising:

(i) formulating a composition comprising:
at least one mRNA expression vector encoding a phenotype sensor; and
a delivery vehicle comprising a cationic lipid, a polyethylenimine (PEI) derivative, a polymer, a polypeptide or peptide, a nanoparticle, or a lipid-based particle;

(ii) administering the composition to a target cell via transfection; and

(iii) detecting the phenotype of the target cell.

44. The method of embodiment 43, wherein the step of detecting the target cell phenotype comprises performing multi-electrode array (MEA) analysis on the cell, measuring action potential profiles and/or Ca2+ transient dynamics, and/or measuring fluorescent cTNT
45. The method of embodiments 43 or 44, wherein the phenotype sensor is selected from the group consisting of an opsin and a protein capable of sensing cell electrophysiology and combinations thereof.
46. The method of embodiment 45, wherein the phenotype sensor is selected from the group consisting of Quasar, Jaws, Catch-V5, ChR2, Archer1, FlicR1, ArcD95H, GCaMP6f, and cTNT-E2Crimson and combinations thereof.
47. The method of any of embodiments 43-46, wherein the at least one mRNA comprises at least one modified nucleotide, at least one untranslated region, and/or at least one mutation resulting in codon optimization.
48. The method of any of embodiments 43-47, wherein the delivery vehicle comprises a cationic lipid comprising a lipofectamine.
49. The method of any of embodiments 43-47, wherein the delivery vehicle comprises a polyethylenimine derivative selected from the group consisting of linear PEI derivatives.
50. The method of any of embodiments 43-47, wherein the delivery vehicle comprises a polymer selected from the group consisting of virus-like polymers.
51. The method of any of embodiments 43-47, wherein the delivery vehicle comprises a nanoparticle selected from the group consisting of viruses and virus-like particles.
52. The method of any of embodiments 43-47, wherein the delivery vehicle comprises a lipid-based particle selected from the group consisting of liposomes and nanoliposomes.

While several possible embodiments are disclosed above, embodiments of the present disclosure are not so limited. These exemplary embodiments are not intended to be exhaustive or to unnecessarily limit the scope of the disclosure, but instead were chosen and described in order to explain the principles of the present disclosure so that others skilled in the art may practice the disclosure. Indeed, various modifications of the disclosure in addition to those described herein will become apparent to those skilled in the art from the foregoing description. Such modifications are intended to fall within the scope of the appended claims. Further, the terminology employed herein is used for the purpose of describing exemplary embodiments only and the terminology is not intended to be limiting since the scope of the various embodiments of the present disclosure will be limited only by the appended claims and equivalents thereof.

All patents, applications, publications, test methods, literature, and other materials cited herein are hereby incorporated by reference in their entirety as if physically present in this specification.

Sequence Listing
Tbox18-containing, synthetic DNA
SEQ ID NO: 1
GGGAAATAAGAGAGAAAAGAAGAGTAAGAAGAAATATAAGAGCCACCATG
GCCGAGAAGAGAAGAGGCAGCCCCTGCTCCATGCTGAGCCTGAAGGCCCA
CGCCTTCAGCGTGGAAGCTCTGATCGGCGCTGAGAAGCAGCAGCAGCTGC
AGAAGAAGAGGCGGAAGCTGGGCGCCGAAGAGGCCGCTAGAGCTGTGGAT
GATGGCGGCTGTTCTAGAGGCGGCGGAGCTGGCGAGAAGGGATCTTCTGA
AGGCGACGAGGGCGCTGCCCTGCCTCCTCCTGCTGGCGCTACATCTGGCC
CTGCTAGAAGCGGCGCTGACCTGGAAAGAGGCGCTGCTGGGGGCTGCGAG
GATGGATTTCAGCAGGGCGCTAGCCCACTGGCTAGCCCTGGCGGATCTCC
TAAGGGCAGCCCTGCCAGATCCCTGGCCAGACCTGGAACACCTCTGCCTA
GCCCTCAGGCCCCTAGAGTGGATCTGCAGGGGGCCGAACTGTGGAAGAGA
TTCCACGAGATCGGCACCGAAATGATCATCACCAAGGCCGGCAGACGGAT
GTTCCCCGCCATGAGAGTGAAGATCAGCGGCCTGGACCCCCACCAGCAGT
ACTATATCGCCATGGACATCGTGCCCGTGGACAACAAGAGATACAGATAC
GTGTACCACAGCAGCAAGTGGATGGTGGCCGGCAACGCCGACTCTCCCGT
GCCTCCTAGAGTGTACATCCACCCTGACAGCCCCGCCAGCGGCGAGACAT
GGATGAGACAAGTGATCAGCTTCGACAAGCTGAAGCTGACCAACAACGAG
CTGGACGACCAGGGCCACATCATCCTGCACAGCATGCACAAGTACCAGCC
CAGAGTGCACGTGATCAGAAAGGACTGCGGCGACGACCTGAGCCCCATCA
AGCCTGTGCCTAGCGGAGAGGGCGTGAAGGCTTTCAGCTTCCCCGAGACA
GTGTTCACCACCGTGACCGCCTACCAGAACCAGCAGATCACCAGACTGAA
GATCGACAGAAACCCCTTCGCCAAGGGCTTCAGAGACAGCGGCAGAAACA
GAATGGGCCTGGAAGCCCTGGTGGAAAGCTACGCCTTTTGGAGGCCCAGC
CTGAGAACCCTGACCTTCGAGGACATCCCCGGCATCCCCAAGCAGGGCAA
CGCCAGCAGTAGCACACTGCTGCAGGGAACCGGAAACGGCGTGCCAGCCA
CACACCCTCATCTGCTGAGCGGCAGCTCTTGCAGCAGCCCAGCTTTTCAC
CTGGGCCCCAACACCAGCCAGCTGTGTTCTCTGGCCCCTGCCGACTACAG
CGCCTGTGCTAGATCTGGCCTGACCCTGAACAGATACAGCACCAGCCTGG
CCGAGACATACAACAGACTGACCAACCAGGCCGGCGAGACTTTCGCCCCT
CCTAGAACCCCTAGCTACGTGGGCGTGTCCAGCAGCACCTCCGTGAACAT
GAGCATGGGCGGCACCGACGGCGACACCTTCAGCTGTCCTCAGACCAGCC
TGTCCATGCAGATCTCCGGCATGAGCCCTCAGCTGCAGTACATCATGCCC
AGCCCTAGCAGCAACGCCTTCGCCACCAACCAGACACACCAGGGCAGCTA
CAACACCTTCCGGCTGCACAGCCCTTGCGCCCTGTACGGCTACAACTTCT
CCACCAGCCCTAAGCTGGCCGCCAGCCCCGAGAAGATCGTGTCTAGCCAG
GGAAGCTTTCTGGGCAGCTCCCCCAGCGGCACCATGACCGATAGACAGAT
GCTGCCCCCCGTGGAAGGCGTGCACCTGCTGTCTAGCGGAGGCCAGCAGA
GCTTCTTCGACAGCAGAACCCTGGGCAGCCTGACACTGAGCAGCAGCCAG
GTGTCCGCCCACATGGTGCCTGCCGCTGCTACAGAGCAGAAGCTGATTAG
CGAAGAGGACCTGGCCGCTAACGACATCCTGGACTACAAGGACGACGACG
ACAAAGTGTGATAAGCTGCCTTCTGCGGGGCTTGCCTTCTGGCCATGCCC
TTCTTCTCTCCCTTGCACCTGTACCTCTTGGTCTTTGAATAAAGCCTGAG
TAGGAAGGC-(poly-A tail)
CatCh-containing, synthetic DNA
SEQ ID NO: 2
GGGAAATAAGAGAGAAAAGAAGAGTAAGAAGAAATATAAGAGCCACCATG
GATTACGGCGGAGCCCTGTCTGCCGTGGGCAGAGAACTGCTGTTCGTGAC
CAACCCCGTGGTCGTGAACGGCAGCGTGCTGGTGCCTGAGGACCAGTGTT
ACTGCGCCGGCTGGATCGAGAGCAGAGGCACAAACGGCGCCCAGACCGCC
TCTAACGTGCTGCAGTGGCTGGCCGCTGGCTTCAGCATCCTGCTGCTGAT
GTTCTACGCCTACCAGACCTGGAAGTCTACCTGCGGCTGGGAAGAGATCT
ACGTGTGCGCCATCGAGATGGTCAAAGTGATCCTGGAATTCTTCTTCGAA
TTCAAAAACCCCTCCATGCTGTACCTGGCCACCGGCCACAGAGTGCAGTG
GCTGAGATACGCCGAGTGGCTGCTGACCTGCCCCGTGATCTGCATCCACC
TGAGCAACCTGACCGGCCTGTCCAACGACTACAGCAGACGGACCATGGGC
CTGCTGGTGTCCGACATCGGCACAATCGTGTGGGGCGCCACAAGCGCCAT
GGCCACAGGCTACGTGAAAGTGATCTTCTTCTGCCTGGGCCTGTGCTACG
GCGCCAACACATTCTTCCACGCCGCCAAGGCCTACATCGAGGGCTACCAC
ACAGTGCCCAAGGGCAGATGCAGACAGGTCGTGACAGGCATGGCCTGGCT
GTTCTTCGTGTCCTGGGGCATGTTCCCCATCCTGTTCATCCTGGGCCCTG
AGGGCTTCGGCGTGCTGTCTGTGTACGGCTCTACCGTGGGCCACACCATC
ATCGACCTGATGAGCAAGAACTGTTGGGGACTGCTGGGCCACTACCTGAG
AGTGCTGATCCACGAGCACATCCTGATTCACGGCGACATCAGAAAGACCA
CCAAGCTGAACATCGGCGGCACCGAGATCGAGGTGGAAACCCTGGTGGAA
GATGAGGCCGAGGCTGGCGCTGTGCCAGCCGCTGCTACAGGTAAGCCTAT
CCCTAACCCTCTCCTCGGTCTCGATTCTACGTGATAAGCTGCCTTCTGCG
GGGCTTGCCTTCTGGCCATGCCCTTCTTCTCTCCCTTGCACCTGTACCTC
TTGGTCTTTGAATAAAGCCTGAGTAGGAAGGC-(Poly-A tail)
FlicR1-containing, synthetic DNA
SEQ ID NO: 3
GGGAAATAAGAGAGAAAAGAAGAGTAAGAAGAAATATAAGAGCCACCATG
GAAGGCTTCGACGGCAGCGATTTTTCCCCTCCTGCCGATCTCGTTGGAGT
TGGCGGAGCCGTGATGAGAAACGTGGTGGACGTGACCATCAACGGCGACG
TGACAGCCCCTCCTAAAGCCGCTCCTAGAAAGTCCGAGAGCGTGAAGAAG
GTGCACTGGAACGACGTGGACCAGGGACCTAGCGAGAAGCCCGAGACACG
GCAAGAGGAAAGAATCGACATCCCCGAGATCAGCGGCCTTTGGTGGGGAG
AGAATGAGCATGGTGTTGGCGGCGGACGGATGGAAATCCCTACAACAGGC
GTGGGCAGAGTGCAGTTTAGAGTGCGGGCCGTGATTGACCACCTGGGCAT
GAGAGCCTTTGGCGTGTTCCTGATCCTGCTGGACATCATCCTGATGATCA
TTGACCTGAGCCTGCCTGGCAAGAGCGAGAGCAGCCAGAGCTTCTATGAT
GGCCTGGCTCTGGCCCTGAGCTGCTACTTCATGCTGGATCTGGGCCTGAG
AATCTTCGCCTACGGACCCAAGAACTTCTTCACAAACCCCTGGGAAGTCG
CCGACGGCCTGATCATCGTGGTCACCTTTGTGGTCACCATCTTCTACACC
GTGCTGGACGAGTACTTCCAAGAGACAGGCGCCGATGGACTGGGACAGCT
GGTTGTTCTGGCTCGGCTGCTGAGAGTCGTCAGACTGGCCAGAATCTTCT
ACTCCCACCAGCAGAGAGTGGTGTCCGAGAGAATGTACCCCGAGGACGGC
GTGCTGAAGTCTGAGATCAAGAAAGGCCTGCGGCTGAAGGACGGCGGACA
CTATGCTGCCGTGGTCAAGACCACCTACAAGGCCAAGAAACCCGTGCAGC
TGCCTGGCGCCTACATCGTGGATATCAAGCTGGACATTGTGTCCCACAAC
GAGGACTACACCATCGTGGAACAGTGCGAGAGAGCCGAGGGCAGACATTC
TACAGGCGGCATGGACGAGCTGTACAAAGGCGGAACAGGCGGCTCCCTGG
TGTCCAAAGGCGAGGAAGTGAACAAGGCCATTATCAAAGAATTCATGCGG
TTCAAGGTCCACATGGAAGGCAGCGTGAACGGCCACGAGTTCGAGATTGA
AGGCGAAGGCGAGGGTAGACCCTACGAGGCCTTTCAGACCGCCAAGCTGA
AAGTGACCAAAGGCGGCCCTCTGCCTTTCGCCTGGGATATCCTGTCTCCT
CAGTTTATGTACGGCAGCAAGGCCTACATCAAGCACCCCGCCGACATTCC
CGACTACTTCAAGCTGAGCTTCCCCGAGGGCTTCAGATGGGAGAGAGTGA
TGAACTTCGAGGATGGCGGCATCATCCACGTGAACCAGGATAGCTCTCTG
CAGGACGGGGTGTTCATCTACAAAGTGAAGCTGCGGGGCACCAACTTTCC
ACCTGATGGCCCTGTGATGCAGAAAAAGACCATGGGCTGGGAAGCCACCA
GACCTGCCGCTGCCACATATCCCTACGACGTGCCAGACTACGCCTGATGA
GCTGCCTTCTGCGGGGCTTGCCTTCTGGCCATGCCCTTCTTCTCTCCCTT
GCACCTGTACCTCTTGGTCTTTGAATAAAGCCTGAGTAGGAAGGCTCGCT
TTCTTGCTGTCCAATTTCTATTAAAGGTTCCTTTGTTCCCTAAGTCCAAC
TACTAAACTGGGGGATATTATGAAGGGCCTTGAGCATCTGGATTCTGCCT
AATAAAAAACATTTATTTTCATTGCGC-(poly-A tail)
Jaws-containing, synthetic DNA
SEQ ID NO: 4
GGGAAATAAGAGAGAAAAGAAGAGTAAGAAGAAATATAAGAGCCACCATG
ACCGCCGTGTCTACCACAGCCACAACAGTGCTGCAGGCCACACAGAGCGA
CGTGCTGCAAGAGATCCAGAGCAACTTCCTGCTGAACAGCAGCATCTGGG
TCAACATTGCCCTGGCCGGCGTTGTGATCCTGCTGTTTGTGGCCATGGGC
CGCGATCTGGAAAGCCCTAGAGCCAAGCTGATCTGGGTCGCCACAATGCT
GGTGCCCCTGGTGTCTATCAGCAGCTATGCCGGACTGGCCTCTGGCCTGA
CAGTGGGCTTTCTGCAAATGCCTCCTGGACACGCCCTGGCTGGACAAGAA
GTTCTGTCTCCCTGGGGCAGATACCTGACCTGGACCTTCAGCACCCCTAT
GATTCTGCTGGCCCTGGGACTGCTGGCCGATACAGATATCGCCAGCCTGT
TCACCGCCATCACCATGGACATCGGGATGTGTGTGACAGGCCTGGCCGCT
GCTCTGATCACAAGCAGCCATCTGCTGAGATGGGTGTTCTACGGAATCAG
CTGCGCCTTCTTCGTGGCCGTGCTGTATGTGCTGCTGGTGCAGTGGCCTG
CCGATGCTGAAGCTGCCGGAACCTCTGAGATCTTCGGCACCCTGAGAATC
CCTGACCGTGGTGTGTGGCTGGGCTACCCTATTCTGTTTGCCCTGGGCTC
TGAAGGCGTGGCACTGCTGTCTGTGGGAGTGACAAGCTGGGGCTATAGCG
GCCTGGACATCCTGGCCAAATACGTGTTCGCCTTTCTGCTCCTCAGATGG
GTCGCCGCCAATGAGGGAACAGTGTCTGGCTCTGGCATGGGCATTGGATC
TGGCGGAGCTGCCCCTGCTGATGATCCAGCTGCTGCCACCTATCCTTACG
ACGTGCCCGACTACGCCTGATGAGCTGCCTTCTGCGGGGCTTGCCTTCTG
GCCATGCCCTTCTTCTCTCCCTTGCACCTGTACCTCTTGGTCTTTGAATA
AAGCCTGAGTAGGAAGGCTCGCTTTCTTGCTGTCCAATTTCTATTAAAGG
TTCCTTTGTTCCCTAAGTCCAACTACTAAACTGGGGGATATTATGAAGGG
CCTTGAGCATCTGGATTCTGCCTAATAAAAAACATTTATTTTCATTGCG
C-(poly-A tail)
Archer1-containing, synthetic DNA
SEQ ID NO: 5
TTTTAAGCTTTAATACGACTCACTATAGGGAAATAAGAGAGAAAAGAAGA
GTAAGAAGAAATATAAGAGCCACCATGGATTACGGCGGAGCCCTGTCTGC
CGTGGGCAGAGAACTGCTGTTCGTGACCAACCCCGTGGTCGTGAACGGCA
GCGTGCTGGTGCCTGAGGACCAGTGTTACTGCGCCGGCTGGATCGAGAGC
AGAGGCACAAACGGCGCCCAGACCGCCTCTAACGTGCTGCAGTGGCTGGC
CGCTGGCTTCAGCATCCTGCTGCTGATGTTCTACGCCTACCAGACCTGGA
AGTCTACCTGCGGCTGGGAAGAGATCTACGTGTGCGCCATCGAGATGGTC
AAAGTGATCCTGGAATTCTTCTTCGAATTCAAAAACCCCTCCATGCTGTA
CCTGGCCACCGGCCACAGAGTGCAGTGGCTGAGATACGCCGAGTGGCTGC
TGACCTGCCCCGTGATCCTGATCCACCTGAGCAACCTGACCGGCCTGTCC
AACGACTACAGCAGACGGACCATGGGCCTGCTGGTGTCCGACATCGGCAC
AATCGTGTGGGGCGCCACAAGCGCCATGGCCACAGGCTACGTGAAAGTGA
TCTTCTTCTGCCTGGGCCTGTGCTACGGCGCCAACACATTCTTCCACGCC
GCCAAGGCCTACATCGAGGGCTACCACACAGTGCCCAAGGGCAGATGCAG
ACAGGTCGTGACAGGCATGGCCTGGCTGTTCTTCGTGTCCTGGGGCATGT
TCCCCATCCTGTTCATCCTGGGCCCTGAGGGCTTCGGCGTGCTGTCTGTG
TACGGCTCTACCGTGGGCCACACCATCATCGACCTGATGAGCAAGAACTG
TTGGGGACTGCTGGGCCACTACCTGAGAGTGCTGATCCACGAGCACATCC
TGATTCACGGCGACATCAGAAAGACCACCAAGCTGAACATCGGCGGCACC
GAGATCGAGGTGGAAACCCTGGTGGAAGATGAGGCCGAGGCTGGCGCTGT
GCCAGCCGCTGCTACAGGTAAGCCTATCCCTAACCCTCTCCTCGGTCTCG
ATTCTACGTGATAAGCTGCCTTCTGCGGGGCTTGCCTTCTGGCCATGCCC
TTCTTCTCTCCCTTGCACCTGTACCTCTTGGTCTTTGAATAAAGCCTGAG
TAGGAAGGCGGCCGCGTCGACAAAAA
Catch-V5-containing, synthetic DNA
SEQ ID NO: 6
TTTTAAGCTTTAATACGACTCACTATAGGGAAATAAGAGAGAAAAGAAGA
GTAAGAAGAAATATAAGAGCCACCATGGATTACGGCGGAGCCCTGTCTGC
CGTGGGCAGAGAACTGCTGTTCGTGACCAACCCCGTGGTCGTGAACGGCA
GCGTGCTGGTGCCTGAGGACCAGTGTTACTGCGCCGGCTGGATCGAGAGC
AGAGGCACAAACGGCGCCCAGACCGCCTCTAACGTGCTGCAGTGGCTGGC
CGCTGGCTTCAGCATCCTGCTGCTGATGTTCTACGCCTACCAGACCTGGA
AGTCTACCTGCGGCTGGGAAGAGATCTACGTGTGCGCCATCGAGATGGTC
AAAGTGATCCTGGAATTCTTCTTCGAATTCAAAAACCCCTCCATGCTGTA
CCTGGCCACCGGCCACAGAGTGCAGTGGCTGAGATACGCCGAGTGGCTGC
TGACCTGCCCCGTGATCTGCATCCACCTGAGCAACCTGACCGGCCTGTCC
AACGACTACAGCAGACGGACCATGGGCCTGCTGGTGTCCGACATCGGCAC
AATCGTGTGGGGCGCCACAAGCGCCATGGCCACAGGCTACGTGAAAGTGA
TCTTCTTCTGCCTGGGCCTGTGCTACGGCGCCAACACATTCTTCCACGCC
GCCAAGGCCTACATCGAGGGCTACCACACAGTGCCCAAGGGCAGATGCAG
ACAGGTCGTGACAGGCATGGCCTGGCTGTTCTTCGTGTCCTGGGGCATGT
TCCCCATCCTGTTCATCCTGGGCCCTGAGGGCTTCGGCGTGCTGTCTGTG
TACGGCTCTACCGTGGGCCACACCATCATCGACCTGATGAGCAAGAACTG
TTGGGGACTGCTGGGCCACTACCTGAGAGTGCTGATCCACGAGCACATCC
TGATTCACGGCGACATCAGAAAGACCACCAAGCTGAACATCGGCGGCACC
GAGATCGAGGTGGAAACCCTGGTGGAAGATGAGGCCGAGGCTGGCGCTGT
GCCAGCCGCTGCTACAGGTAAGCCTATCCCTAACCCTCTCCTCGGTCTCG
ATTCTACGTGATAAGCTGCCTTCTGCGGGGCTTGCCTTCTGGCCATGCCC
TTCTTCTCTCCCTTGCACCTGTACCTCTTGGTCTTTGAATAAAGCCTGAG
TAGGAAGGCGGCCGCGTCGACAAAAA
ChR2-containing, synthetic DNA
SEQ ID NO: 7
ttttaagcttTAATACGACTCACTATAGGGAAATAAGAGAGAAAAGAAGA
GTAAGAAGAAATATAAGAGCCACCATGGATTACGGCGGAGCCCTGTCTGC
CGTGGGCAGAGAACTGCTGTTCGTGACCAACCCCGTGGTCGTGAACGGCA
GCGTGCTGGTGCCTGAGGACCAGTGTTACTGCGCCGGCTGGATCGAGAGA
GAGGCACAAACGGCGCCCAGACCGCCTCTAACGTGCTGCAGTGGCTGGCC
GCTGGCTTCAGCATCCTGCTGCTGATGTTCTACGCCTACCAGACCTGGAA
GTCTACCTGCGGCTGGGAAGAGATCTACGTGTGCGCCATCGAGATGGTCA
AAGTGATCCTGGAATTCTTCTTCGAATTCAAAAACCCCTCCATGCTGTAC
CTGGCCACCGGCCACAGAGTGCAGTGGCTGAGATACGCCGAGTGGCTGCT
GACCTGCCCCGTGATCCTGATCCACCTGAGCAACCTGACCGGCCTGTCCA
ACGACTACAGCAGACGGACCATGGGCCTGCTGGTGTCCGACATCGGCACA
ATCGTGTGGGGCGCCACAAGCGCCATGGCCACAGGCTACGTGAAAGTGAT
CTTCTTCTGCCTGGGCCTGTGCTACGGCGCCAACACATTCTTCCACGCCG
CCAAGGCCTACATCGAGGGCTACCACACAGTGCCCAAGGGCAGATGCAGA
CAGGTCGTGACAGGCATGGCCTGGCTGTTCTTCGTGTCCTGGGGCATGTT
CCCCATCCTGTTCATCCTGGGCCCTGAGGGCTTCGGCGTGCTGTCTGTGT
ACGGCTCTACCGTGGGCCACACCATCATCGACCTGATGAGCAAGAACTGT
TGGGGACTGCTGGGCCACTACCTGAGAGTGCTGATCCACGAGCACATCCT
GATTCACGGCGACATCAGAAAGACCACCAAGCTGAACATCGGCGGCACCG
AGATCGAGGTGGAAACCCTGGTGGAAGATGAGGCCGAGGCTGGCGCTGTG
CCAGCCGCTGCTACAGGTAAGCCTATCCCTAACCCTCTCCTCGGTCTCGA
TTCTACGTGATAAgctgccttctgcggggcttgccttctggccatgccct
tcttctctcccttgcacctgtacctcttggtctttgaataaagcctgagt
aggaaggcggccgcgtcgacaaaaa
Quasar-containing, synthetic DNA
SEQ ID NO: 8
TTTTAAGCTTTAATACGACTCACTATAGGGAAATAAGAGAGAAAAGAAGA
GTAAGAAGAAATATAAGAGCCACCATGGATTACGGCGGAGCCCTGTCTGC
CGTGGGCAGAGAACTGCTGTTCGTGACCAACCCCGTGGTCGTGAACGGCA
GCGTGCTGGTGCCTGAGGACCAGTGTTACTGCGCCGGCTGGATCGAGAGC
AGAGGCACAAACGGCGCCCAGACCGCCTCTAACGTGCTGCAGTGGCTGGC
CGCTGGCTTCAGCATCCTGCTGCTGATGTTCTACGCCTACCAGACCTGGA
AGTCTACCTGCGGCTGGGAAGAGATCTACGTGTGCGCCATCGAGATGGTC
AAAGTGATCCTGGAATTCTTCTTCGAATTCAAAAACCCCTCCATGCTGTA
CCTGGCCACCGGCCACAGAGTGCAGTGGCTGAGATACGCCGAGTGGCTGC
TGACCTGCCCCGTGATCCTGATCCACCTGAGCAACCTGACCGGCCTGTCC
AACGACTACAGCAGACGGACCATGGGCCTGCTGGTGTCCGACATCGGCAC
AATCGTGTGGGGCGCCACAAGCGCCATGGCCACAGGCTACGTGAAAGTGA
TCTTCTTCTGCCTGGGCCTGTGCTACGGCGCCAACACATTCTTCCACGCC
GCCAAGGCCTACATCGAGGGCTACCACACAGTGCCCAAGGGCAGATGCAG
ACAGGTCGTGACAGGCATGGCCTGGCTGTTCTTCGTGTCCTGGGGCATGT
TCCCCATCCTGTTCATCCTGGGCCCTGAGGGCTTCGGCGTGCTGTCTGTG
TACGGCTCTACCGTGGGCCACACCATCATCGACCTGATGAGCAAGAACTG
TTGGGGACTGCTGGGCCACTACCTGAGAGTGCTGATCCACGAGCACATCC
TGATTCACGGCGACATCAGAAAGACCACCAAGCTGAACATCGGCGGCACC
GAGATCGAGGTGGAAACCCTGGTGGAAGATGAGGCCGAGGCTGGCGCTGT
GCCAGCCGCTGCTACAGGTAAGCCTATCCCTAACCCTCTCCTCGGTCTCG
ATTCTACGTGATAAGCTGCCTTCTGCGGGGCTTGCCTTCTGGCCATGCCC
TTCTTCTCTCCCTTGCACCTGTACCTCTTGGTCTTTGAATAAAGCCTGAG
TAGGAAGGCGGCCGCGTCGACAAAAA
FLAG tag
SEQ ID NO: 9
DYKDDDDK

1. A composition comprising:
a messenger RNA (mRNA) encoding a protein selected from the group consisting of a differentiation factor, a transcription factor, and a phenotype sensor; and
a delivery vehicle configured to deliver the mRNA to a target cell.

2. The composition of claim 1, wherein the mRNA encodes a differentiation factor selected from the group consisting of Tbx18, Tbx3, Tbx5, SHOX2, and combinations thereof.

3. The composition of claim 1, wherein the mRNA encodes a phenotype sensor selected from the group consisting of an opsin, a protein capable of sensing cell electrophysiology, and combinations thereof.

4. (canceled)

5. The composition of claim 1, wherein the mRNA comprises at least one of a modified nucleotide, a cap, an untranslated region, a polyadenine tail, and a mutation resulting in codon optimization.

6. The composition of claim 1, wherein the delivery vehicle comprises a cationic lipid, a polyethylenimine (PEI) derivative, a polymer, a polypeptide or peptide, a nanoparticle, or a lipid-based particle.

7. The composition of claim 1, wherein the delivery vehicle comprises a polyethylenimine derivative selected from the group consisting of linear PEI derivatives, a cationic lipid comprising a lipofectamine, a polymer selected from the group consisting of virus-like polymers, a nanoparticle selected from the group consisting of viruses and virus-like particles, or a lipid-based particle selected from the group consisting of liposomes and nanoliposomes.

8.-10. (canceled)

11. The composition of claim 1 further comprising a small molecule tethered to the mRNA.

12. The composition of claim 11, wherein the small molecule is selected from the group consisting of inhibitors of innate immune sensors.

13. A method comprising:
administering the composition of claim 1 to a target cell; and
optionally detecting the phenotype of the target cell.

14. The method of claim 13, wherein the method is for modulating cell phenotypes; and
wherein the mRNA encodes a differentiation factor selected from the group consisting of Tbx18, Tbx3, Tbx5, SHOX2, and combinations thereof.

15. The method of claim 13, wherein the mRNA encodes a phenotype sensor selected from the group consisting of an opsin, a protein capable of sensing cell electrophysiology, and combinations thereof.

16. The method of claim 15, wherein the phenotype sensor is selected from the group consisting of Quasar, Jaws, Catch-V5, ChR2, Archer1, FlicR1, ArcD95H, GCaMP6f, cTNT-E2Crimson, and combinations thereof.

17. The method of claim 13, wherein the mRNA comprises at least one of a modified nucleotide, a cap, an untranslated region, a polyadenine tail, and a mutation resulting in codon optimization.

18. The method of claim 16, wherein the delivery vehicle comprises a cationic lipid comprising a lipofectamine.

19. The method of claim 16, wherein the delivery vehicle comprises a polyethylenimine derivative selected from the group consisting of linear PEI derivatives.

20. The method of claim 16, wherein the delivery vehicle comprises a polymer selected from the group consisting of virus-like polymers.

21. The method of claim 16, wherein the delivery vehicle comprises a nanoparticle selected from the group consisting of viruses and virus-like particles.

22. The method of claim 16, wherein the delivery vehicle comprises a lipid-based particle selected from the group consisting of liposomes and nanoliposomes.

23. The method of claim 16 further comprising a small molecule tethered to the at least one mRNA.

24. The method of claim 23, wherein the small molecule is selected from the group consisting of inhibitors of innate immune sensors.

25. The method of claim 16, wherein the target cell is selected from the group consisting of a cardiac cell, a cardiomyocyte, a neuronal cell, a cell located within the eye, a pancreatic cell, a PSC, an IPSC, an ESC, and a PSC cardiomyocyte.

26. The method of claim 16, wherein the target cell is a cardiac cell, a cardiomyocyte, a PSC, an IPSC, an ESC, or an PSC cardiomyocyte; and
wherein the phenotype of the target cell has been modified to cause the target cell to differentiate into one or more of an atrial myocyte, a ventricular myocyte, and a cardiac pacemaker cell.

27. The method of claim 13, wherein the phenotype of the target cell has been modified to cause the target cell to differentiate into a cardiac pacemaker cell; and
wherein the method further comprises providing to a subject in need thereof differentiated cardiac pacemaker cell.

28. (canceled)

29. The method of claim 13, wherein:
the method is for treating and/or preventing a cardiac disorder in a subject in need thereof;
the mRNA encodes a phenotype sensor;
the delivery vehicle comprises a cationic lipid, a polyethylenimine (PEI) derivative, a polymer, a polypeptide or peptide, a nanoparticle, or a lipid-based particle;
administering comprises administering via transfection the composition to the target cell;
the target cell is selected from the group consisting of a cardiac cell, a cardiomyocyte, a PSC, an IPSC, an ESC, and a PSC cardiomyocyte;
the method comprises detecting the phenotype of the transfected target cell by detecting the activity of the phenotype sensor; and
the method further comprises re-implanting the transfected target cell in the subject.

30. The method of claim 29, wherein the cardiac disorder is selected from the group consisting of atrioventricular block, sick sinus syndrome, and other arrhythmias which typically require the implantation of a pacemaker device.

31. The method of claim 30, wherein detecting the phenotype of the transfected target cell comprises one or more of:
performing multi-electrode array (MEA) analysis on the target cell;
measuring action potential profiles;
measuring Ca2+ transient dynamics; and
measuring fluorescent cTNT.

32.-42. (canceled)

43. The method of claim 13, wherein:
the method is for determining a cell phenotype;
the mRNA is an mRNA expression vector encoding a phenotype sensor;
the delivery vehicle comprises a cationic lipid, a polyethylenimine (PEI) derivative, a polymer, a polypeptide or peptide, a nanoparticle, or a lipid-based particle;
administering comprises administering via transfection the composition to the target cell; and
the method comprises detecting the phenotype of the target cell.

44. The method of claim 43, wherein detecting the phenotype of the transfected target cell comprises one or more of:
performing multi-electrode array (MEA) analysis on the target cell;
measuring action potential profiles;
measuring Ca2+ transient dynamics; and
measuring fluorescent cTNT; and
wherein the phenotype sensor is selected from the group consisting of an opsin, a protein capable of sensing cell electrophysiology, and combinations thereof.

45. (canceled)

46. The method of claim 44, wherein the phenotype sensor is selected from the group consisting of Quasar, Jaws, Catch-V5, ChR2, Archer1, FlicR1, ArcD95H, GCaMP6f, cTNT-E2Crimson, and combinations thereof.

47.-52. (canceled)

53. The composition of claim 1 further comprising a small molecule tethered to the mRNA;
wherein the mRNA encodes a phenotype sensor selected from the group consisting of an opsin, a protein capable of sensing cell electrophysiology, and combinations thereof; and
wherein the delivery vehicle comprises:
a polyethylenimine derivative selected from the group consisting of linear PEI derivatives;
a polymer selected from the group consisting of virus-like polymers; or
a lipid-based particle selected from the group consisting of liposomes and nanoliposomes.

54. The composition of claim 53, wherein the phenotype sensor is selected from the group consisting of Quasar, Jaws, Catch-V5, ChR2, Archer1, FlicR1, ArcD95H, GCaMP6f, cTNT-E2Crimson, and combinations thereof; and
wherein the small molecule is selected from the group consisting of inhibitors of innate immune sensors.
